(19) United States
(12) Patent Application Publication
(10) Pub. No.: US 2002/0004586 A1
(43) Pub. Date: Jan. 10, 2002

(54) Prion-binding activity in serum and proteins

(76) Inventors: Adriano Aguzzi, Zurich (CH); Michael Boris Fischer, Dubendorf (CH)

Correspondence Address: MARSHALL, O'TOOLE, GERSTEIN, MURRAY & BORUN, 6300 SEARS TOWER, 233 SOUTH WACKER DRIVE, CHICAGO, IL, 60606-6402, US

(21) Appl. No.: 09/840,341

(22) Filed: Apr. 23, 2001

Related U.S. Application Data
(63) Continuation-in-part of application No. 09/407,667, filed on Sep. 28, 1999.

(30) Foreign Application Priority Data
Mar. 27, 2001 (EP) PCT/EP01/03481
Jun. 26, 2000 (IB) PCT/IB00/00849

(51) Int. Cl. C07K 14/435; C07K 16/18; G01N 33/53

(52) U.S. Cl. 530/388.2; 530/380; 435/7.1

(57) ABSTRACT

Disclosed are methods and tools for the concentration and detection as well as quantification of pathological prion proteins as well as agents to be used in said detection and/or in the prevention or treatment of prion diseases. Said agents are factors with prion binding activities found in blood serum and blood plasma.

### FIELD OF THE INVENTION

The present invention concerns a method and agents to detect transmissible spongiform encephalopathies as well as agents for the prevention and treatment of respective infections.

### BACKGROUND ART

According to all available evidence, the agents causing transmissible spongiform encephalopathies, termed prions, are devoid of informational nucleic acids and consist of an “infectious” protein (termed PrP^{Sc}) capable of converting a normal host protein called PrP^{C} into a likeness of themselves. The only organ system in which histopathological damage and its clinical sequelae can be demonstrated as a consequence of infection with prions is the nervous system (Brandner et al., 1996). This consideration applies to both the human transmissible spongiform encephalopathies, such as Creutzfeldt-Jakob disease, Gerstmann-Sträussler-Scheinker Syndrome, Kuru and fatal familial insomnia, and all known prion encephalopathies of animals (Weber and Aguzzi, 1997). The latter comprise scrapie in sheep, bovine spongiform encephalopathy, and chronic wasting diseases of mule, deer and exotic ungulates (Weissmann and Aguzzi, 1997).

However, there is no doubt that prions, herewith operationally defined as the infectious agents causing transmissible spongiform encephalopathies, can colonize organs other than the central and peripheral nervous system, and can be demonstrated in extracerebral compartments (Aguzzi et al., 1997). The problem of which organ systems can harbour infectivity is further complicated by the existence of prion strains. Just like strains of conventional viruses, prions can come in various different flavors, each one of which has its specific preferences with regard to the host range which is infectible and also to the type of cells in which it replicates (Aguzzi, 1998). One paradoxical situation, which is of immediate relevance to the question of blood safety, is exemplified by the radically different organ tropism of the BSE agent in cows and in humans. BSE prions seem to be largely confined to the neural compartment of cows, even after oral exposure (Wells et al., 1998). A very accurate study of the pathogenesis of experimental BSE in cows upon feeding 100 grams of infected brain has disclosed that there is only a short and transient period during which infectivity can be demonstrated in the terminal ileum (Wells et al., 1998). At later time points, BSE prions can only be shown in brain, spinal cord, and dorsal root ganglia. The exact localization of BSE in the terminal ileum is not known. It is being discussed whether infectivity resides in Peyer's patches or in the neural compartment which comprises the Plexus submucosus Meissner and the Plexus myentericus Auerbach. There is a great body of circumstantial evidence that BSE prions can provoke new variant Creutzfeldt-Jakob Disease (nvCJD) (Bruce et al., 1997; Chazot et al., 1996; Hill et al., 1997; Will et al., 1996), but no absolutely final evidence has been produced. For the purpose of the following discussion we will regard the evidence that BSE and new variant Creutzfeldt-Jakob Disease caused by the same agent as sufficiently verified (Aguzzi and Weissmann, 1996). Upon passage into humans, and consecutive progression to manifest nvCJD, prions experience a dramatic shift in their organotropism. Instead of remaining confined mainly to neural structures, they can be detected in many organs belonging to the immune system, including most notably tonsils, spleen, and as recently demonstrated, the appendix (Hilton et al., 1998). It is, therefore, unavoidable to conclude that the tropism of the infectious agent for various structures depends on both the strains of prions in question (and therefore it is in part autonomous to its carrier) and on the species in which prion disease manifests itself (Aguzzi and Weissmann, 1998).

These considerations are not only of academic interest. In fact, the transmissibility of the agent by iatrogenic manipulations (i.e. blood transfusions, organ transplants, etc.) is crucially affected by such parameters.

Horizontal Transmissibility of Human Prions:

Prion diseases of humans are undoubtedly transmissible. However, transmission is achieved only under particular circumstances. One could say that in this respect prion diseases fulfill the characteristics of transmissibility delineated by Semmelweiss for puerperal fever: these affections are infectious but not contagious. Direct transmissions of brain-derived material from a patient suffering from Creutzfeldt-Jakob disease to other persons have documentedly resulted in transmission of disease. A particularly tragic case occurred in the early seventies in Zurich, when electrodes used for cortical recordings from Creutzfeldt-Jakob patients were sterilized (formaldehyde and alcohol) and used in additional patients. Disease was transmitted to the very young recipients (Bernoulli et al., 1977). Also, transplantation of cornea has most likely resulted in transmission of disease (Duffy et al., 1974).

Despite these tragic dimensions, cases of iatrogenic transmission of CJD via neurosurgical procedures have remained rather rare. This is not totally understood, given that the frequency of subclinical CJD must be much higher than that of manifest disease, and that most neurosurgical instruments are not sterilized in a way that would reliably inactivate prions. Therefore, the quite rare nature of iatrogenic transmission is likely to indicate that host factors, in addition to the virulence of prions, may affect the probability that infection takes place. This notion is strengthened by the epidemiology of iatrogenic CJD (iCJD) upon transmission of contaminated dura mater. It has been estimated that several thousands patients, predominantly in Japan, may have been exposed to the CJD agent via preparations of cadaveric dura mater which had been contaminated with prions. However, it appears that less then 2% of those exposed have developed disease so far. While we can rejoice about this low efficiency in the “take” of infectivity, we do not fully understand the biological basis for the apparent protection enjoyed by most subjects exposed to CJD prions. The largest problem with iatrogenic transmission has occurred as result of administration of pituitary hormones of cadaveric origin (Gibbs et al., 1985). Preparations of growth hormone and of gonadotropins contaminated with human prions have caused the death of more then 80 persons, predominantly children. Due to the long latency that can be expected when the agent is introduced into extracerebral sites, such as via intramuscular injection, it must be assumed that further cases from this procedure, which has been stopped more than a decade ago, will arise in the future.

Besides its tragic human dimension and the harm that it has cost to the patients and to their physicians, the pituitary hormone disaster needs to be understood in detail, because the anterior lobe of the pituitary gland is not a part of the central nervous system. Therefore, these events may serve as a paradigm for transmission of prions via contaminated extracerebral tissue that does not belong to the canonical sites of replication of prions. The observation that latency after intracerebral contamination is much shorter than latency after peripheral infection is in good agreement with experimental data from various animal models, and suggest that a rather lengthy phase of extracerebral events (which may include replication of the agent, and invasion of specific extraneuronal systems) may be a precondition to prion neuroinvasion (Aguzzi, 1997).

Factors influencing the neurotropism of prions:

There is good reason to suspect that neuroinvasive processes in the course of prion infections are very tightly controlled. Perhaps the best argument in this respect derives from the observation that the incubation times of experimental animals inoculated intraperitoneally with scrapie prions are extremely reproducible. Upon inoculation with a known amount of standard inoculum, the experience in various laboratories has been that latencies between inoculation and first clinical symptoms display standard variations in the order of only a few percent points (Klein et al., 1997). If prion neuroinvasion were a totally random process, one would expect a large variability in the incubation times, which would depend on processes governed by chance. However, if some rate-limiting processes control neuroinvasion, these may be responsible for the remarkable precision of the incubation times. Indeed, we very much hope that this interpretation is correct because if such processes exist they might be amenable to manipulation, which in turn may represent a post-exposure strategy to prevent overt prion disease. Indeed, various mechanisms have been explored by which neuroinvasions may be accomplished.

A first phase or neuroinvasion seems to be widespread colonization of the immune system. This colonization can be visualized by homogenizing spleen, lymph nodes, tonsils, and also appendix, and injecting the homogenates into suitable experimental animals. The dilution of the homogenates at which 50% of the experimental animals become sick, contains one ID50 of the infectious agent in each inoculum.

The second phase of neuroinvasion seems to be dependent upon a compartment which cannot be replaced by adoptive bone marrow transfer (Blattler et al., 1997) and which may be represented by the peripheral nervous system and/or the follicular dendritic cells resistant to germinal center of secondary lymphatic organs. It appears that this second compartment necessitates the expression of normal prion protein in order to support neuroinvasion (Blattler et al., 1997).

Neuroinvasion is dependent on a functional immune system, and immunodeficient mice do not develop disease after inoculation with a moderate dose of the agent (Fraser et al., 1996; Kitamoto et al., 1991; Lasmezas et al., 1996; O'Rourke et al., 1994). One crucial component of the immune system necessary for neuroinvasion has been traced to the physical presence of terminally mature B-lymphocytes To date, it is not clear whether B cells are required because they bind physically prions and carry them to sites of neuroinvasion, or whether B cells produce factors, or induce processes, which are indirectly responsible for facilitating neuroinvasion (Klein et al., 1997). Given the requirement for B-lymphocytes secreting lymphotoxin for the maturation of follicular dendritic cells, and the fact that follicular dendritic cells accumulate large amounts of scrapie prions in experimental situations, it is tempting to speculate that the main function of B-lymphocytes in the aforementioned process consists in allowing FDCs to mature.

The cellular and molecular basis of prion neuroinvasion:

Following experimental inoculation of mice with prions at peripheral sites, there is typically a prolonged, clinically silent replication phase of the infectious agent within the lymphoreticular system (LRS). This occurs prior to detectable neuroinvasion by prions and the subsequent occurrence of neurological symptoms. During this preclinical latency period, prions may replicate to high titers within lymphoreticular tissues. Elucidating the cell types in which prions replicate within the peripheral lymphoid tissue and —crucially—how prions are transported to the central nervous system (CNS) is of great interest and clinical importance. Despite considerable evidence implicating the role of the immune system in peripheral prion pathogenesis, there have been few studies on the identity of the cells involved in this process. It has been shown many years ago that whole-body irradiation of mice with gamma rays fails to influence prion pathogenesis or incubation time of scrapie. This has been taken as an argument against significant involvement of proliferating cells in the lymphoreticular phase of prion propagation. Instead, follicular dendritic cells (FDC) have been considered as the prime cell type for prion replication within lymphoid tissue since PrP^{Sc} accumulates in the follicular dendritic network of scrapie infected wild-type and nude mice (Kitamoto et al., 1991). In addition, severe combined immuno deficient mice (SCID), which lack mature B- and T-cells, and which do not appear to have functional FDCs, are highly resistant to scrapie after intraperitoneal inoculation and fail to replicate prions in the spleen (Fraser et al., 1996; Kitamoto et al., 1991; Lasmezas et al., 1996; O'Rourke et al., 1994). Interestingly, bone-marrow reconstitution of SCID mice with wild-type spleen cells restores full susceptibility to scrapie after peripheral infection (Fraser et al., 1996; Klein et al., 1998). These findings suggest that an intact, or at least partially functional, immune system comprising lymphocytes and FDC is required for efficient transfer of prions from the site of peripheral infection to the CNS.

The time course for the development of scrapie disease following intracerebral or intraperitoneal inoculation is highly reproducible and is primarily dependent on the dose of the inoculum. Therefore, neuroinvasion by prions migrating from peripheral lymphoid tissue may depend on tightly controlled, rate-limiting reactions. In order to identify such rate-limiting steps during prion neuroinvasion, PrPC deficient mice bearing PrP-overexpressing cerebral neurografts were infected intraperitoneally (i.p.). No disease was observed in the grafts, suggesting that neuroinvasion depends on PrP expression in extracerebral sites. This was further underlined by reconstitution of the lymphoid system with PrPC expressing cells, which restores infectivity in the lymphoid tissue, but still fails to transport prions to the nervous system.

As prions can be detected in lymphoreticular tissues, an understanding of the peripheral pathogenesis is of immediate importance in assessing risks of iatrogenic transmission of human BSE via exposure to blood or tissues from preclinical cases, and possibly from contaminated surgical instruments, or even blood and blood products. Additionally, such advances might pave the way for the development of sensitive diagnostic tests and the means to block prion neuroinvasion. Why is contamination of the blood supply with prions an important issue? The main problem is new variant CJD. For one thing, we by far do not know as much about the epidemiology and iatrogenic transmissibility of this new disease as we do for sporadic CJD (sCJD). What is most unsettling, the distribution of preclinical disease in Great Britain and possibly in other countries is very obscure, and the little knowledge that is being gathered is far from reassuring (Will et al., 1999). Moreover, there is all reason to believe that nvCJD may be much more “lymphoinvasive” than its sporadic counterpart. In particular, nvCJD prions can be easily detected in lymphatic organs such as tonsils and appendix (Hill et al., 1999; Hill et al., 1997; Hilton et al., 1998), a fact that was previously demonstrated to be true for scrapie (Schreuder et al., 1997; Schreuder et al., 1998; Vankeulen et al., 1996), but not for sCJD prions. While all available evidence points to follicular dendritic cells as the prion reservoir in lymphatic organs, splenic lymphocytes of experimentally inoculated mice can be infected with prions (Raeber et al., 1999). Although prion infectivity of circulating lymphocytes appear to be at least two logs lower than that detected in splenic lymphocytes (Raeber et al., 1999), the possibility that circulating lymphocytes may be in equilibrium with their splenic siblings call for cautionary measures. The nature of the latter is still matter of controversy and debate: leukodepletion has been advocated, but at present there is no certainty about its efficacy, and even whether the presently available technologies for leukoreduction are necessary and/or sufficient for decreasing the threat to blood supply that derives from nvCJD. In addition, it has to be taken into account that, even if blood prion infectivity were to be originally contained in lymphocytes in vivo, lysis of cells may lead to contamination of non-particulate fractions and, in the absence of appropriate measures of removal, of stable blood products.

The second consideration applies so secondary prophylaxis. Given the very large numbers of infectious BSE material that has entered the human food chain, it is possible that many individuals harbor preclinical nvCJD. It is imperative and urgent to develop strategies that will help control spread of the agent and that will hopefully prevent the clinical outbreak of symptoms in these persons. Possible targets for the interference with neuroinvasion are rate-limiting processes that control prion replication within the infected individual. In light of the knowledge discussed above, treatments that target the neuro-immune interface of prion replication and neuroinvasion (Aguzzi and Collinge, 1997) seem a promising area for research aimed at post-exposure prophylaxis.

Methods to detect prions and their limitation:

In the age of real-time kinetic polymerase chain reaction (PCR), we have become very spoiled with respect to the detection thresholds which we demand from assays geared at detecting viral contaminants in blood. Consider the case of HIV: here the introduction of quantitative PCR technologies has pushed the limit of detection in blood and blood products down to quasi-perfection. Even when PCR techniques have not proved that useful, or have not yet met with such widespread acceptance, ultrasensitive immunochemical methods, such as time-resolved fluorescent ELISA, have progressed to a degree of sophistication that is highly satisfactory for most screening application. So why do we still have a problem with prion detection in blood?

The most formidable problem derives from the unique biology of the prion. According to more-or-less accepted wisdom, infectious prions are likely to consist solely of the PrP^{Sc} protein, which has exactly the same amino acid structure as the normal cellular protein PrP^{C}. A more noncommittal way of wording this fact would be to state that PrP^{Sc} is the only known surrogate marker for prion infectivity: this latter statement is likely to be agreeable upon by both the proponents of the protein-only hypothesis and by those who still believe that the infectious agent is a virus.

The consequence of the fact mentioned above for prion detection is obvious: if prion-specific nucleic acids do not exist, any PCR-based screening assay to detect said nucleic acid will not be an option. Therefore, we are left with immunochemical assays. Besides being less sensitive than PCR by several orders of magnitude, these are also fraught with a series of prion-specific problems. The biggest trouble, again, derives directly from the peculiar biology of TSE agents. As explained above, PrP^{Sc} possesses the same chemical composition as PrP^{C}, and the latter is a membrane-bound protein that is normally found in many cell types of healthy individuals including white blood cells (Aguzzi and Weissmann, 1997). Although PrP^{C} and PrP^{Sc} differ in a number of physical properties, it appears to be extremely difficult to develop immunological reagents which reliably differentiate between these two isoforms. Only one monoclonal antibody has been described to react with PrP^{Sc} but not with PrP^{C}, and its practical usefulness remains to be demonstrated since fourteen months after its publication no follow-up studies have appeared and even the company which developed this reagent in the first place does not appear to use it in its in-house screening assay for BSE prions.

The hitherto best method for the detection of prions is by performing Western blot analysis with homogenized brain tissue that has been digested with proteinase K (PK). The digestion is necessary since for Western blot analysis the secondary structure is broken up so that no difference is found any more between cellular prions (PrP^{C}) and pathological prions (PrP^{Sc}), however, while PrP^{C} is readily digested by PK under specified conditions, PrP^{Sc} is only degraded to relatively large fragments called PrP²⁷⁻³⁰.

It is also already known to concentrate proteins by adsorbing them to so called magnetic beads (MB) to which a specific antibody is bound. However, the application of such a concentration method to PrP has been assumed to be impossible due to the specific features of prions.

Thus, still a great need exists to have a sensitive method or test to detect small amounts of prions of the PrP^{Sc} confirmation not only for diagnosis but also for further investigating the disease, as well as agents to perform such tests.

### BRIEF DESCRIPTION OF THE INVENTION

Hence it is one object of the present invention to provide a method for the detection of the pathological prion protein as PrP^{Sc} or PrP²⁷⁻³⁰, respectively.

It is another object of the present invention to provide a method for searching for prion in particular PrP^{Sc} interacting agents.

Still another object of the present invention are agents specifically recognizing PrP^{Sc} and/or PrP²⁷⁻³⁰.

Still another object of the present invention are solid phase materials such as e.g. magnetic beads carrying such agents and composition comprising same.

Still another object of the present invention are compositions comprising such agents for purifying body fluids and sterilization of surgical and diagnostic instruments.

Still another object is to provide an improved method for diagnosing transmissible spongiform encephalopathies (TSE) and means therefor.

PrP^{Sc} is also termed “infectious protein”. PrP^{Sc} means a prion protein with a confirmation which differs from the “normal” confirmation of PrPc in healthy organisms which do not show or develop any signs of TSE.

A “prion binding site which selectively interacts with PrP^{Sc} but not with PrP^{C}” means a molecule or part of a molecule which can bind to PrP^{Sc} but fails to bind to PrP^{C}. Such a binding site can be provided e.g. by a low molecular organic compound, a peptide or protein as well as by antigen binding sites of antibodies, wherein said term “antibodies” interalia, comprises conventional antibodies, scFv-fragments (Fab) and (Fab₂) fragments. The term “selectively” in this context means that a compound having the said binding site reacts at least twofold, preferably at least fivefold, preferably at least tenfold, stronger with the prion protein in the PrP^{Sc} confirmation than with the prion protein in the PrP^{c} confirmation. Most preferably, the binding site shows at least the selectivity for PrP^{Sc} as shown by plasminogen.

“A factor with prion binding activity” means a compound which can bind to a prion protein and carries the selective prion binding site of the invention. The factor can be a low molecular compound but preferably is a peptide of at least 10 amino acids length or a protein, which, however, can carry further nonprotein residues such as carbohydrate residues or lipid residues. Said factor can be e.g. of animal or human origin or synthetic.

“Selective prion binding site as contained in plasminogen” means a binding site as provided by a peptide or protein having all or part of the amino acid sequence as contained in a plasminogen of animal or human origin which selectively interacts with PrP^{Sc} but not with PrP^{c}.

“Derivative of plasminogen plasminogen” means a peptide or protein which carries at least one amino acid addition, substitution or deletion compared to the naturally occurring plasminogen or fragment thereof but still capable of selectively interacting with PrP^{Sc} and not with PrP^{c}. Such derivatives can easily be prepared, e.g. by site directed mutagenesis of the nucleic acid encoding the naturally occurring plasminogen or by peptide synthesis.

“Fragment of plasminogen” means a part of a naturally occurring plasminogen which part is capable of selectively interacting with PrP^{Sc} and not with PrP^{c}.

“Carboxy terminus of PrP^{Sc}” means the first . . . amino acids from the carboxy terminus of a prion protein.

“Ligand” means a compound which selectively binds to the complex of PrP^{Sc} with the binding factor according to the invention but does not interact with free PrP^{Sc} or free factor alone. Such a ligand can be e.g. an antibody or another receptor type protein.

The present invention interalia relates to a method for the concentration of PrP^{Sc} or digestion products thereof, wherein a body fluid or fluidized organ is treated with solid phase material, the material at least partly carries a prion binding site which selectively interacts with PrP^{Sc} but not with PrP^{c}. The selectivity of a given binding site can easily be determined as further described herein below. The sufficient selectivity is shown, e.g., by plasminogen.

In this context, “fluidized organ” means tissue derived from an organ and solubilized by mechanical procedures, sonication, or other procedures in order to bring into solution or suspension a significant proportion of its constituents.

In a further preferred embodiment the fluidized organ is a homogenized tissue preferably of the central nervous system, preferably homogenized brain tissue.

In a further preferred method, the body fluid, such as blood, plasma, serum, urine, or lymph is treated with a proteinase, preferably with proteinase K (PK). In a further preferred method the selective prion binding site is contained in a factor with prion binding activity (PrPB). Such factor is preferably a peptide or protein. In case of the peptide or protein, it is of sufficient length to allow stable binding to PrP^{Sc} or the digestion product thereof. A sufficient binding stability (or affinity) is shown, e.g. by plasminogen.

In a further preferred method the solid phase material carries a PrPB as can be found in blood serum or blood plasma, preferably as contained in fraction II of the ammonium sulfate precipitation of serum or plasma.

In a further preferred method the solid phase material carries a PrPB as can be found in plasminogen, fibrinogen or in plasma fraction I or ammonium sulfate precipitation.

In a further preferred method the PrPB is selected from plasminogen, fibrinogen, sPrPBII and ₚPrPBII or fragments thereof which fragments show the same selectivity as the complete protein.

The present invention further relates to a method for the detection and optionally quantification of PrP^{Sc} or digestion products thereof which method comprises the step of selectively binding PrP^{Sc} or the digestion product thereof to a prion binding site as defined above.

In a preferred embodiment the prion binding site is contained in a factor with prion binding activity.

In a preferred embodiment, the factor is selected from factors as contained in blood serum, blood plasma, serum or plasma fractions II of ammonium sulfate precipitation, plasma fraction I of ammonium sulfate precipitation or as provided by plasminogen, fibrinogen, sPrPBII and ₚPrPBII or fragments thereof which show a similar selectivity as the complete protein.

The detection of the produced complex between PrP^{Sc} and the prion binding site can be done by any method currently applicable to the detection of such protein complexes, e.g., by any type of affinity assays or in particular immunoassays as, e.g., described in “The Immunoassay Handbook” of David Wild, Second Edition ISPN0-33-72306-6, Nature Publishing Group. In order to achieve high sensitivity, a fluorescence detection method is preferred.

The present invention further provides for the first time a composition comprising a PrP^{Sc} or digestion product thereof and a material carrying a prion binding site which selectively interacts with PrP^{Sc} but not with PrP^{c}. According to a preferred embodiment, the prion binding site is contained in a factor with prion binding activity, preferably selected from a factor as contained in blood serum, blood plasma, serum or plasma fraction II of ammonium sulfate precipitation, plasma fraction I of ammonium sulfate precipitation or as contained in plasminogen, fibrinogen, sPrPBII and ₚPrPBII.

The present invention further provides a solid phase material which carries a prion binding site of the invention, which binding site is preferably contained in a factor with prion binding activity which factor is preferably a factor selected from a factor as contained in plasma serum, blood plasma, serum or plasma fraction II of ammonium sulfate precipitation, plasma fraction I of ammonium sulfate precipitation or as contained in plasminogen, fibrinogen, sPrPBII and ₚPrPBII.

The present invention further provides a protein complex which comprises a PrP^{Sc} and a factor with a prion binding site which selectively interacts with PrP^{Sc} but not PrP^{c}, preferably selective PrP^{Sc} a binding site as contained in plasminogen.

The present invention further provides a test kit for the detection of pathological prion protein such as PrP^{Sc}, in body fluids or organs such as blood, urine, cerebrospinal fluid, brain tissue, lymph nodes, tonsils which kit comprises a factor with a prion binding site according to the invention and the factor is preferably selected from a factor as contained in blood serum, blood plasma, serum or plasma fraction II of ammonium sulfate precipitation, plasma fraction I of ammonium sulfate precipitation or as contained in plasminogen, fibrinogen, sPrPBII and ₚPrPBII.

The present invention further provides an assay for the diagnosis of human transmissible human spongiform encephalopathies or prion encephalopathies of animals comprising the step of contacting the sample to be tested with a prion binding site which selectively interacts with PrP^{Sc} but not with PrP^{c}. In a preferred embodiment the sample is derived from blood. The tested animal or human is diagnosed positive, i.e. for running risk of developing a transmissible spongiform encephalopathy if PrP^{Sc} can be detected in the tested sample.

The present invention for the first time allows the detection of PrP^{Sc} in a sample which may contain also normal PrP^{c} by using a binding factor for PrP^{c} which contains the selective prion binding site according to the invention.

In a further aspect of the invention, the carboxy terminal part of PrP^{Sc} is the target for the selective binding site. It has surprisingly been found that the carboxy terminus of PrP^{Sc} provides a binding site which allows discrimination between PrP^{Sc} and PrP^{c}, which binding site is a preferred target for the binding sites according to the present invention.

It has further surprisingly been found that the binding sites according to the present invention strongly discriminate between PrP^{Sc} and PrP^{c} of different species although it is known that sequence variations exist between prion proteins from different species. Therefore, PrP^{Sc} of different species can be detected by the use of a single prion binding site according to the invention. A very much preferred binding site is the binding site as contained in human plasminogen. It was surprisingly found that the plasminogen binding site interacts with disease associated prion protein from species. The findings according to the invention further suggest that a property common to PrP^{Sc} of various species rather than the peculiarities private to the specific protein structure of individual PrP^{Sc} molecules are responsible for binding to the binding sites in plasminogen.

As a carrier for use in accordance with the present invention, essentially all materials can be considered that are currently available to perform biological assays for detecting a given compound in a biological sample. Such a carrier includes magnetic beads, filter stripes, walls of microtiter plates.

According to one aspect of the present invention, the detection of PrP^{Sc} could be done “indirectly” in that first a complex is formed between PrP^{Sc} and the binding site according to the present invention and the so-formed complex is then selectively detected by a ligand, such as an antibody. Such a ligand which selectively interacts with an epitope formed by the interaction of PrP^{Sc} and its binding factor is also highly selective and detects such a complex only but neither detects free PrP^{Sc} nor free factor according to the invention only.

The products according to the invention are useful to provide new diagnostic kits which contain all ingredients for performing assays in order to detect PrP^{Sc}. Such kits may contain in addition to the products according to the invention buffers, reagents for detecting a product being the result of the presence of PrP^{Sc} in a sample, the working instructions on how to reliably perform an assay.

Since PrP^{Sc} as such is an essential element in the development of TSE reducing the availability of free PrP^{Sc} can be a useful means to avoid and or reduce the speed of BSE development. A means for reducing free PrP^{Sc} is the binding of PrP^{Sc} to a binding site according to the invention, which binding site hence can form part of the pharmaceutical composition.

Alternatively, the binding site according to the invention can be used for removing PrP^{Sc} from a biological bacteria, e.g., in the form of a dialysis wherein blood of the animal to be tested is continuously brought into contact with a carrier containing the binding site of the invention.

The present invention hence allows for the first time a reliable method for diagnosing human transmissible spongiform encephalopathies and prion encephalopathies of animals in which method the material of the animal to be tested is brought into contact with the binding site according to the invention.

In a preferred method of the present invention for the concentration of PrP^{Sc} or digestion products thereof, a body fluid, such as e.g. blood, urine, cerebrospinal fluid etc., or fluidized organ, such as brain tissue, lymph nodes, tonsils etc., is treated with a solid phase material such as magnetic beads (MB) whereby at least part of said material or beads, respectively, carries a prion binding site. A preferred prion binding site is a factor with prion binding activity (PrPB).

The method works very well with a fluidized organ, in particular homogenized tissue of central nervous system, preferably homogenized brain tissue.

In cases where the prion binding site can only distinguish PrP^{C} and PrP^{Sc} in digested form, it is necessary to digest the fluid or fluidized organ prior to the actual concentration step. A suitable digestion is obtained by digestion with proteinase K (PK), whereby it is important to inactivate the proteinase K prior to the addition of the solid phase, e.g. MBs.

Methods are, however, preferred which do not require the digestion of the starting material in order to achieve selectivity for the PrP^{Sc}.

Preferred solid phase materials carrying PrPB can be prepared by coupling such materials with blood serum, or blood plasma, such as fresh frozen plasma of mammals, whereby an excess of protein is present during the coupling procedure. Such factors are designated spPrPB (s= serum and p= plasma) (see below). Even more preferred solid materials carrying PrPB are prepared by coupling purified plasminogen or fibrinogen or fragments thereof to solid phase such as the beads(see below).

Very suitable solid materials are magnetic beads since they can easily be treated with specific components of interests and easily be collected by applying a magnetic field.

A further preferred method of the present invention concerns the detection (and optionally quantification) of PrP^{Sc} or digestion products thereof, wherein PrP^{Sc} is first concentrated as described above, optionally also with previous digestion of the fluid or fluidized organ, and then detected and optionally compared with a standard. A suitable detection method is Western blot analysis. Such test may furthermore be embodied by other detection methods such as a microtiter plate format immunoassay (e.g. ELISA assay), an immunoprecipitation assay, a BIACORE assay, immunocytochemical assay, histoblot assay etc.

Besides of the above mentioned methods, the present invention also concerns factors with prion binding activities such as sPrPBII, which is a prion binding activity in fraction II of ammonium sulfate precipitation of serum or ₚPrPBII which is a factor with prion binding activity in fraction II of ammonium sulfate precipitation of normal or fresh frozen plasma. Said factors are of course subject matter of the present invention in any form, such as in isolated form, or as ingredient in a composition, e.g. in a fraction of ammonium sulfate precipitation.

Said factors can be obtained by concentration and/or isolation of PrPBs whereby serum or plasma is subjected to fractionated ammonium sulfate precipitation thus that a PrPB of interest is precipitated, preferably in only one fraction. A further purification can be obtained by the application of further protein isolation methods.

The factors of the present invention are not only suitable for the detection of prions, in particular PrP^{Sc}, but they have further applications in methods for the purification and removal of pathological prion protein from body fluids and organs, such as blood, urine, cerebrospinal fluid, brain tissue, lymph nodes, tonsils etc., or for the sterilization of surgical and/or diagnostic tools, basing on the affinity of PrPB for the pathological prion protein. They are furthermore tools for a therapy regimen based on the modulation of production of PrPB for preventing the spread of prions in the body. Especially suitable in this respect is plasminogen, that is also especially suitable for the purification of body fluids, e.g. blood units. Such purification may e.g. be performed by treating fluids with PrPBIp, in particular with immobilized plasminogen or plasma fractions containing same.

Also part of the present invention is a test for the detection of pathological prion protein such as PrP^{Sc} in body fluids or organs such as blood, urine, cerebrospinal fluid, brain tissue, lymph nodes, tonsils etc, that utilizes the specific binding properties of PrPB to pathological prion protein. Such test can be embodied as a microtiter plate format immunoassay, e.g. ELISA assay, an immunoprecipitation assay, a BIACORE assay, immunogytochemical assay, histoblot assay etc.

Also the DNA sequences specific for biosynthesis of PrPB are comprised by the present invention as well as vectors able to express such DNA sequences in suitable hosts.

Furthermore comprised by the present invention are: a method for purification of PrPB by using PrP²⁷⁻³⁰ as bait; monoclonal and polyclonal antibodies produced in animals such as mice, rabbits, chicken etc., and directed against PrPB; single-chain Fv fragments and other types of fragments of antibodies produced in recombinant phages or in other recombinant systems, and directed against PrPB; a test predictive of susceptibility to prion diseases based on polymorphisms of PrPB, or on variations in the strength and pattern of production of PrPB; a transgenic animal, e.g. mouse that overproduces PrPB in brain, lymph nodes, or other organs, to be used in a bioassay for prions; a knockout animal, in particular a mouse, which is devoid of PrPB, to be used in a bioassay for prions; a production method of PrPB by expressing a DNA sequence specific for the biosynthesis of PrPB in a suitable host cell, such as bacteria, yeast, fungi, or eukaryotic cells, and by purification of PrPB from the aforementioned organisms; a use of natural or synthetic, preferably purified PrPB as a medicament for therapeutical applications in humans and animals; a vaccination of organisms with natural or synthetic PrPB, in particular plasminogen; a diagnostic assay for human and/or animal diseases resulting from abnormal production and/or metabolism of PrPB.

### BRIEF DESCRIPTION OF THE DRAWINGS

FIG. 1 is a scheme showing the IAP method.

FIG. 2 shows Western Blots and IAP experiments of dilution experiments, whereby lanes 1 to 6 and 10 represent usual Western Blots and lanes 7 to 9 and 11 to 13 represent immuno affinity purification (IAP).

FIG. 3 is a scheme showing the prion affinity assay (PAA) method.

FIG. 4 represents Western Blots showing positive and begative controls of the PAA.

FIG. 5 shows the observation that beads coated with sheep anti mouse IgG Abs by DYNAL bind PrPSc but not PrP27-30. Upon preincubation with normal mouse serum PrP27-30 is also bound.

FIG. 6 represents Western Blots showing the results with serum proteins that are coupled to beads. The * means that the coupling was performed in the presence of an excess of proteins.

FIG. 7 shows the effect of the addition of PK-treated brain homogenate to the assay.

FIG. 8 represents Western Blots showing the results with PrP-deficient material.

FIG. 9 represents Western Blots showing PAA of ammonium sulfate precipitates.

FIG. 10 represents Western Blots showing PAA of ammonium sulfate precipitates that are not covalently crosslinked to the beads.

FIG. 11 shows the result of the PAA of the 58 fractions of human plasma that were obtained by chromatography and differential precipitation and tested for binding activity.

FIG. 12 represents Western Blots showing the results with purified plasminogen and fibrinogen.

FIG. 13 represents Western Blots showing the calcium dependency of the binding activity of plasminogen and fibrinogen.

FIG. 14 represents Western Blots showing the dependency of the binding activity of plasminogen on the native state of the proteins.

FIG. 15 represents Western Blots showing PAA of plasminogen that is not covalently crosslinked to the beads.

FIG. 16 shows the concepts of the bioassay.

FIG. 17 shows the results of the bioassay.

FIG. 18 shows PrP^{Sc} binding activity of plasminogen to different species.

FIG. 19 shows precipitation of human PrP^{CJD} by plasminogen.

### DETAILED DESCRIPTION F THE INVENTION

As already mentioned above, there is a great need for a detection method for low concentrations of PrP^{Sc} that can be used as a diagnostic test for transmissible spongiform encephalopathies (TSEs).

There are basically three diagnostic principles for TSEs: histopathological detection of the typical spongiform changes in the CNS, detection of the scrapie-specific isoform of the prion protein, and the bioassay that detects infectivity. All these methods have limitations: histopathology is not useful for preclinical diagnosis since the structural changes appear late in the incubation period. Detection of the scrapie the Western specific is form of prion protein is more sensitive but still much less sensitive than the bioassay. The bioassay can, in principle, detect as little as 1 infectious unit but can last months or even years.

The hitherto used Western blot technique is based on the partial protease resistance of PrP^{Sc} that allows to distinguish between Prp^{C} and PrP^{Sc}. After protease treatment, PrP²⁷⁻³⁰ —the protease resistant core of PrP^{Sc} —can be detected but not PrP^{C} which is completely digested.

Although due to the “stickiness” of prions it was generally assumed that immuno affinity purification (IAP) cannot be applied, it has now been found that concentration can be achieved by applying magnetic beads (MB) carrying a prion binding site, preferably a factor with prion protein binding activity (PrPB).

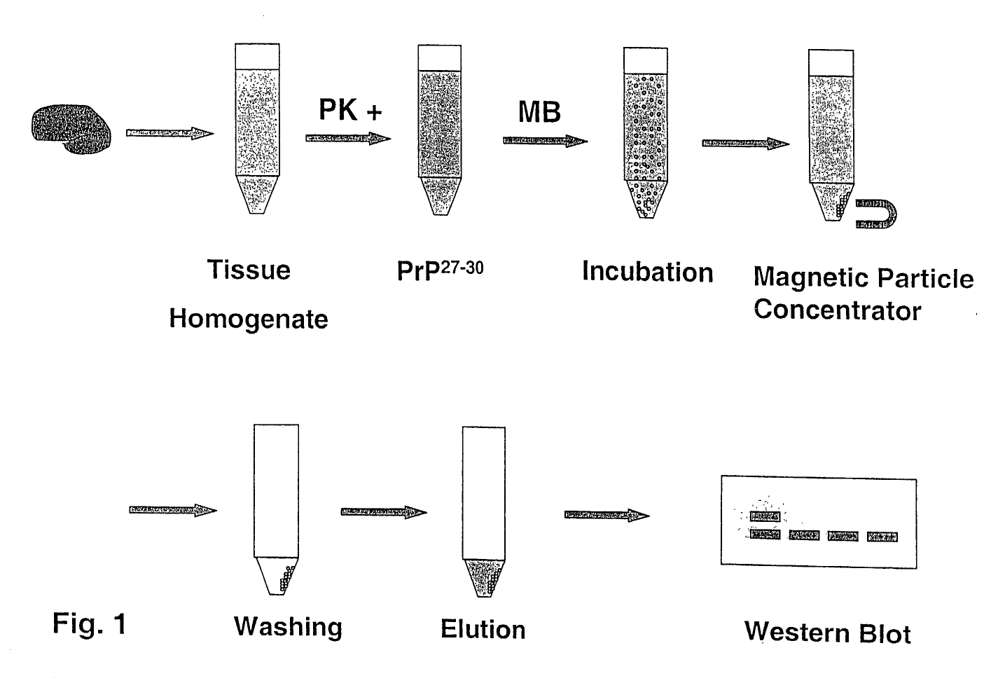

Thus, because the sensitivity of detection of absolute amounts of PrP²⁷⁻³⁰ is a function of antibody affinity, and cannot be easily increased for each given antibody, in the scope of the present invention, despite of the hitherto assumed problems, first an “immuno affinity purification” (IAP) assay has been developed, using antibodies covalently crosslinked to solid phase material, e.g. magnetic beads. Because the monoclonal antibody (6H4 purchased at Prionics, Zurich, Switzerland, described in Korth et al., 1997), originally used for the development of the IAP, is not able to distinguish between PrP^{C} and PrP^{Sc} (it binds both undigested forms as well as digested PrP^{Sc}, i.e. PrP²⁷⁻³⁰), it is necessary to perform Proteinase K digestion prior to the IAP (see FIG. 1).

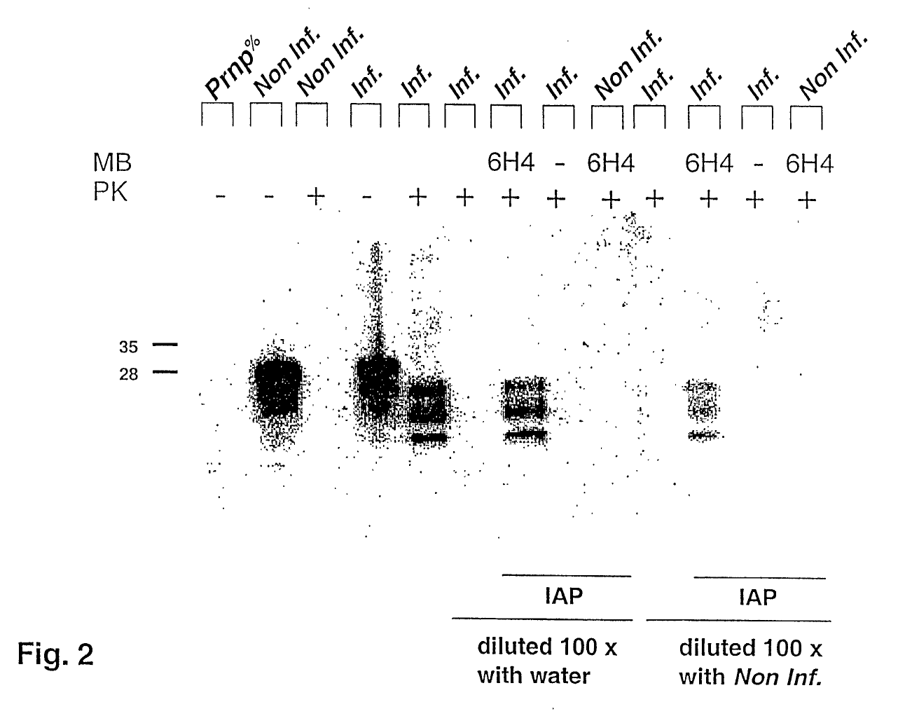

For the development of the present IAP method, the following model system was used: Two tests were performed to determine the efficiency of the method. On the one hand, small amounts of a scrapie-infected mouse brain homogenate were diluted with water and then subjected to the PrP^{Sc} concentration method. On the other hand, small amounts of a scrapie-infected brain homogenate was diluted with brain homogenate of non-infected mice in order to simulate a real situation in which a brain homogenate contains low amounts of PrP^{Sc} (see FIG. 2).

In FIG. 2, lanes 1 to 6 and 10 represent usual Western Blots and lanes 7 to 9 and 11 to 13 represent immuno affinity purification (IAP). PrnP % is material from PrP deficient mice. MB are of course only used for IAP whereby 6H4 refers to MB coupled with 6H4 antibodies and − refers to uncoupled MBs. PRP^{C} refers to brain homogenate of non-infected mice and PrP^{Sc} refers to brain homogenate of scrapie-infected mice. PK refers to Proteinase K digestion whereby − refers to non digestion and + to digested homogenate. The same abbreviations are used for the following figures.

For prion analysis in homogenate, in particular of brain tissue, it is important to use in a first homogenation step low concentration of ionic detergent, followed by low speed centrifugation, preferably 500 g 30 minutes, 4° C. applied twice. For following steps high concentration of non-ionic detergent is used and a protein concentration of the homogenate of at most 5 mg/ml.

Conditions for the proteinase K digestion are preferably 50 μg/ml PK, 37° C. and at least half an hour.

Suitable incubation conditions for the beads with homogenate are e.g. about 1.5 hours at room temperature, whereby for low concentrations longer incubation times might be preferable.

The concentration step in said first attempt was carried out by adding to digested homogenate magnetic beads (MB) carrying said 6H4.

If a digestion step is needed, it has to be performed prior to the concentration step, whereby the digestion, usually by proteinase K, has to be stopped prior to the concentration step by deactivating the proteinase e.g. with phenyl methyl sulfonyl fluoride or another agent known to the skilled person.

By applying the method of the present invention for e.g. brain tissue homogenate, PrP²⁷⁻³⁰ can be concentrated up to amounts detectable by Western blot analysis from tissue comprising much less pathological prion protein than needed for the hitherto known tests.

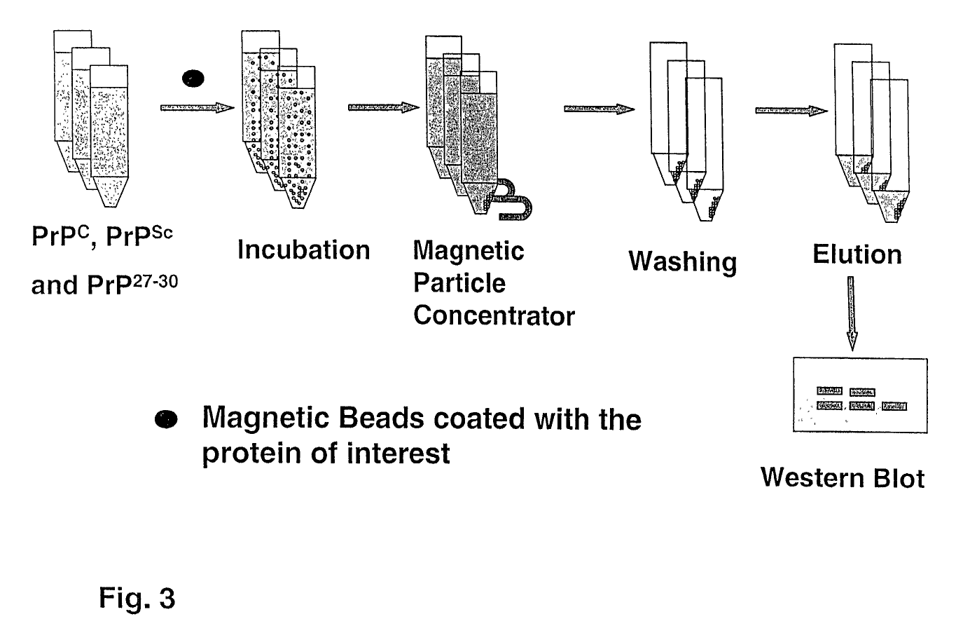

Using largely the same procedure, the above described method can also be applied as prion affinity assay (PAA) by exchanging the monoclonal antibody 6H4 by other substances to be examined, for example in order to find a binding partner for PrP^{Sc} (see FIG. 3).

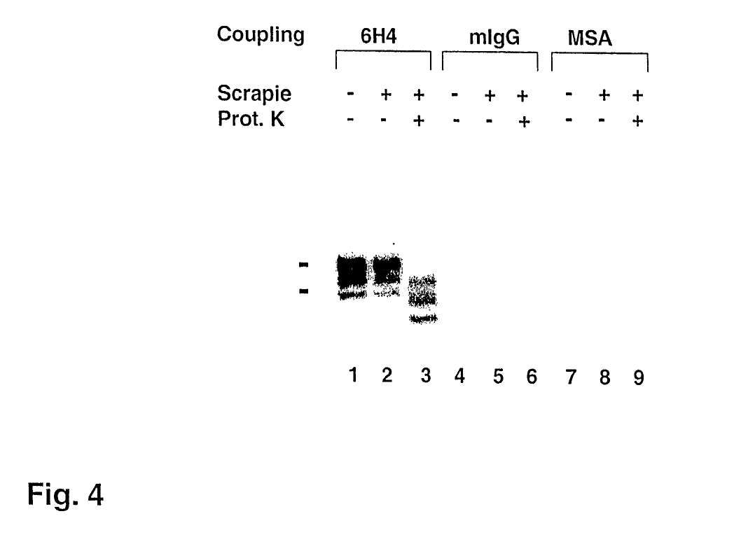

As a positive control of this assay 6H4 (see FIG. 4, lanes 1-3) is used and as a negative control mouse IgG or mouse albumin (see FIG. 4, lanes 4-9).

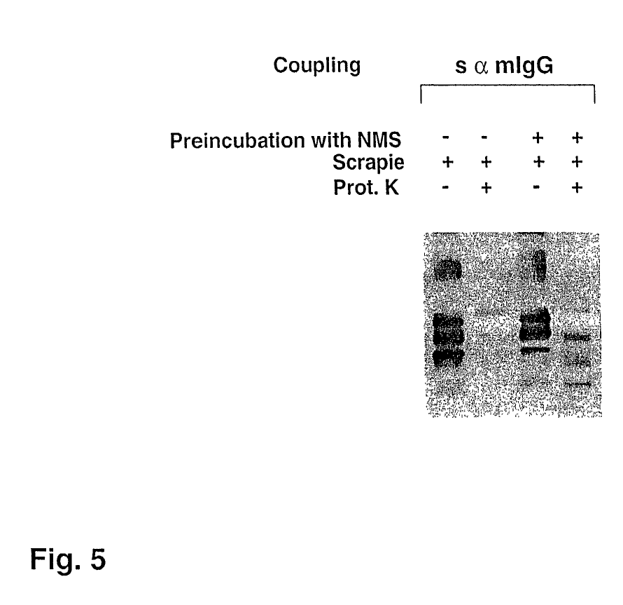

In order to investigate whether a given mouse serum containes IgG that specifically recognize PrP^{Sc} magnetic beads that are already coated by the company DYNAL with sheep antibodies directed against mouse IgGs were used after preincubation with mouse serum. These beads —used without preincubation —were the first negative control (see FIG. 5, lanes 1-2). As a second negative control these beads preincubated with normal mouse serum were used in order to show that IgGs from normal mouse serum do not bind to any form of PrP (see FIG. 5, lanes 3-4). Surprisingly the beads alone showed an affinity to PrP^{Sc} but not to PrP²⁷⁻³⁰. Upon preincubation with normal mouse serum also PrP²⁷⁻³⁰ is bound. Therefore it was hypothised that the sheep antibodies from DYNAL recognize a molecule that is associated with PrP^{Sc} but digested away after PK-treatment. As PrP²⁷⁻³⁰ is bound upon preincubation with normal mouse serum, this serum might contain the molecule with affinity to PrP^{Sc}.

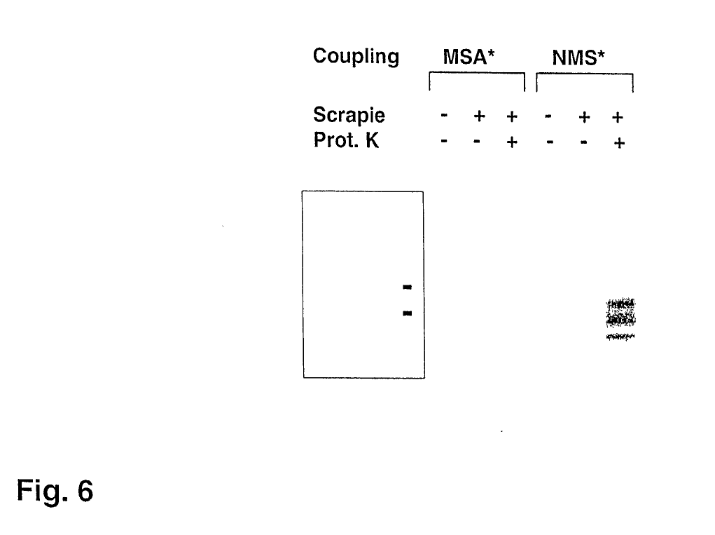

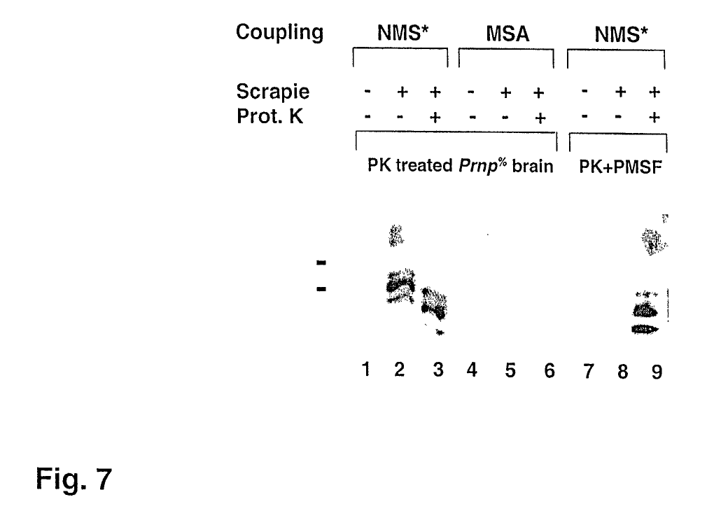

The beads coupled to total mouse serum proteins did not show any affinity to any form of PrP. However, if the coupling of the total serum was performed in the presence of an excess of protein the beads showed the same binding to PrP²⁷⁻³⁰ as the monoclonal antibody 6H4 (see FIG. 6, lanes 4-6) whereas the beads that were coupled in the presence of an excess of albumine still did not show any affinity to any form of PrP (see FIG. 6, lanes 1-3). Though it was not possible to measure any difference of the coupling efficency of the two conditions it might be that offering an excess of proteins causes a sponge on the surface of the beads that binds PrP²⁷⁻³⁰. We also checkled whether PK-treated brain homogenate might enhance the binding as in the case of bound PrP²⁷⁻³⁰ total PK-digested brain homogenate is present: the addition of PK-digested brain homogenate from wild-type C57BL/6 mice or Prnp % mice allowed to bind PrP^{Sc} in addition to PrP²⁷⁻³⁰ (see FIG. 7, lanes 1-3); the addition of inactive PK had no influence on the binding activity (see FIG. 7, lanes 7-9). If coupled in the presence of an excess the activity of binding PrP²⁷⁻³⁰ was also found in the serum of man, sheep, cow and in the serum of terminally scrapie-sick C57BL/6 mice (data not shown).

Apart from an artefact it might well be that serum of several species contains activities (collectively termed PrP^{B}) that interact specifically with the pathogenic isoform of the prion protein and that are kinetically favoured in binding to the beads. The affinity to PrP²⁷⁻³⁰ could then be understood assuming that native PrP^{Sc} present in sick mice is saturated with PrP^{B} which might be released upon proteolytic digest. Alternatively, partial proteolysis may expose PrP^{B} binding sites on PrP^{Sc}. However, the fact that the addition of PK-treated brain homogenate allows to bind PrP^{Sc} indicates that there might be several different interactions leading to our observations.

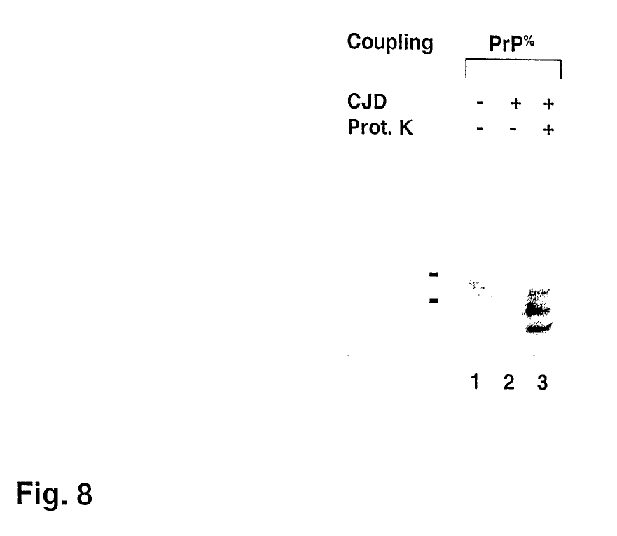

The template-directed refolding hypothesis predicts that PrP^{C} and PrP^{Sc} form heterodimers during the conversion process. Therefore we investigated whether PrP^{B} is identical with PrP^{c}. However, when coupling in excess PrP^{B} activity was present in the serum of Prnp % mice at levels similar to those of wild-type mice, implying that PrP^{c} does not contribute to the binding activity (see FIG. 8).

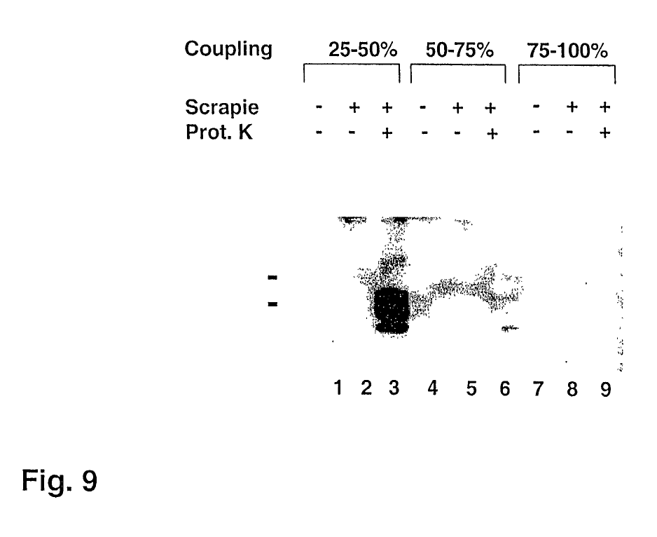

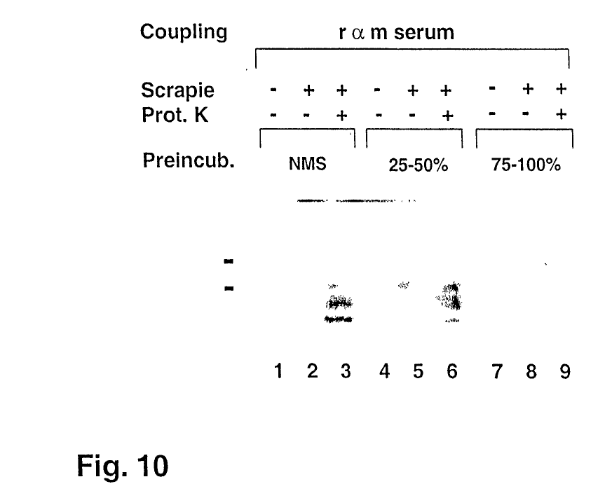

If PrP^{B} activity is not only caused by the special coupling conditions, it should be possible to “purify” it by fractionating mouse serum by differential ammonium sulfate precipitation. Indeed, it was possible to precipitate PrP^{B} at an ammonium sulfate saturation below 50% whereby coupling of each fraction was performed in the presence of an excess of protein (see FIG. 9). While purified rabbit immunoglobulins against total mouse serum did not contain PrP^{B} (data not shown), they efficiently bound PrP²⁷⁻³⁰ upon preincubation with full mouse serum (see FIG. 10, lanes 1-3) or with proteins precipitating between 25% and 50% ammonium sulfate saturation (see FIG. 10, lanes 4-6). Preincubation with proteins precipitating between 75% and 100% ammonium sulfate saturation did not lead to PrP^{B} activity (see FIG. 10, lanes 7-9). This finding is important as it shows that the PrP^{B} activity is a property of one or more serum proteins independent of the covalent crosslink to the surface of the beads.

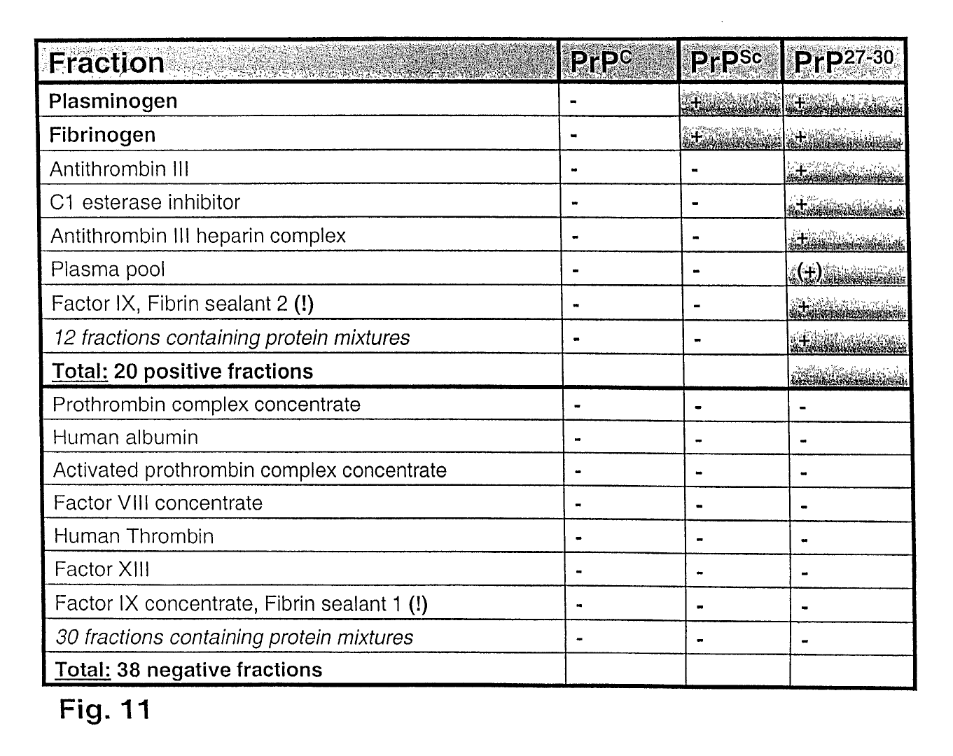

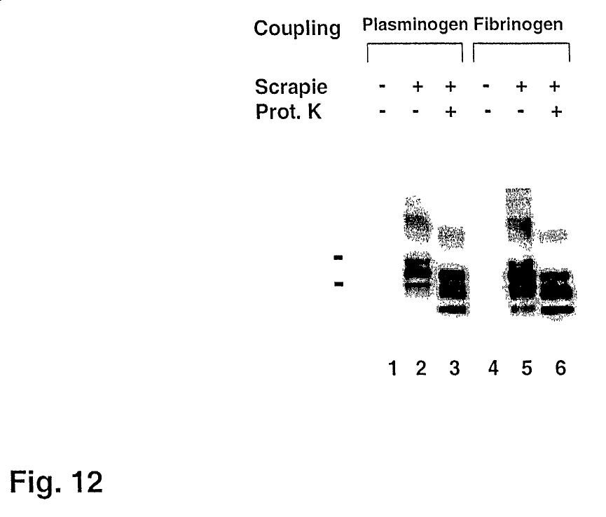

As the ammonium sulfate fractionation worked with human serum as well (data not shown), 58 fractions of human plasma were obtained by chromatography and differential precipitation and tested for binding activity to form an idea of the identity of PrP^{B}. All fractions were not coupled in the presence of an excess of proteins. Therefore the results can directly be compared with 6H4 or mouse IgG. 20 fractions tested positive: Plasminogen, fibrinogen, antithrombin III, antithrombin III heparin complex, C1 esterase inhibitor, factor IX and several fractions containing protein mixtures (see FIG. 11). Purified plasminogen and also purified fibrinogen bound PrP^{Sc} in addition to PrP²⁷⁻³⁰ (see FIG. 12). Out of the 38 fractions that tested negative, 6 contained purified proteins: Prothrombin complex concentrate, albumin, activated prothrombin complex concentrate, factor XIII and thrombin.

As mentioned, there are some hints that the binding of PrP²⁷⁻³⁰ is caused by different effects. The activity that binds PrP²⁷⁻³⁰ is termed spPrP^{B} (s=serum and p=plasma) as it is present in serum and in plasma. Said activity is comparable to the activity found for plasminogen and fibrinogen. Plasminogen and fibrinogen were furthermore characterized as they both bind also PrP^{Sc}.

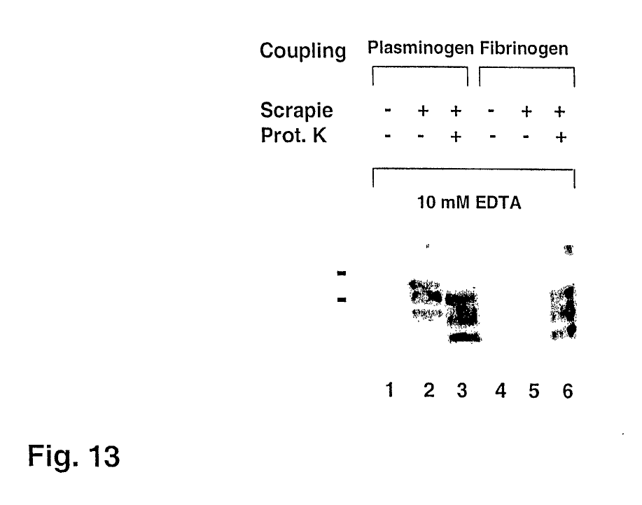

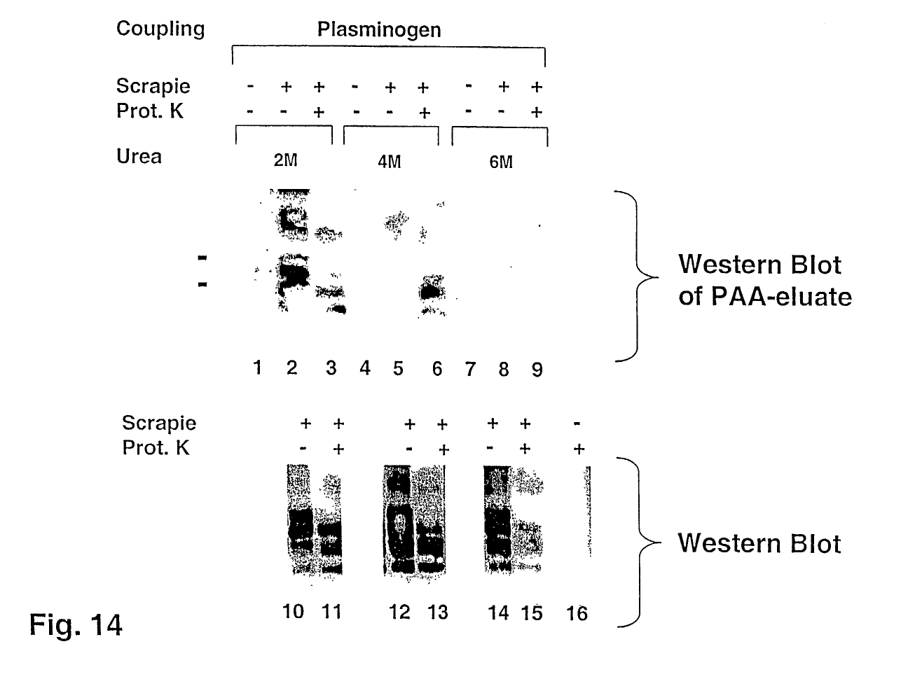

As calcium is an important cofactor in the coagulation cascade it was investigated whether PrP^{B} activity is still intact if coagulation is inhibited by complexing calcium. In the presence of 10 mM EDTA the pathogenic PrP^{Sc} and PrP²⁷⁻³⁰ were still bound by plasminogen (see FIG. 13, lanes 1-3) but only PrP²⁷⁻³⁰ by fibrinogen (see FIG. 13, lanes 4-6). At least in the case of plasminogen this finding speaks against the possibility that the PrP^{B} activity is due to unspecific coagulation. Because PrP^{B} selectively interacts with the pathogenic PrP but not with PrP^{c}, interaction may be conformation-specific. When the assay was carried out in the presence of 6 M urea the fraction containing purified plasminogen didn't bind PrP^{Sc} nor PrP²⁷⁻³⁰ (see FIG. 14, lanes 8-9) under these conditions PrP^{Sc} becomes protease-sensitive (see FIG. 14, lanes 14-15). As the conformation of PrP^{Sc} is thought to be responsible for the PK resistancy we conclude from this experiment that the interaction of plasminogen and PrP^{Sc} is conformation-dependant.

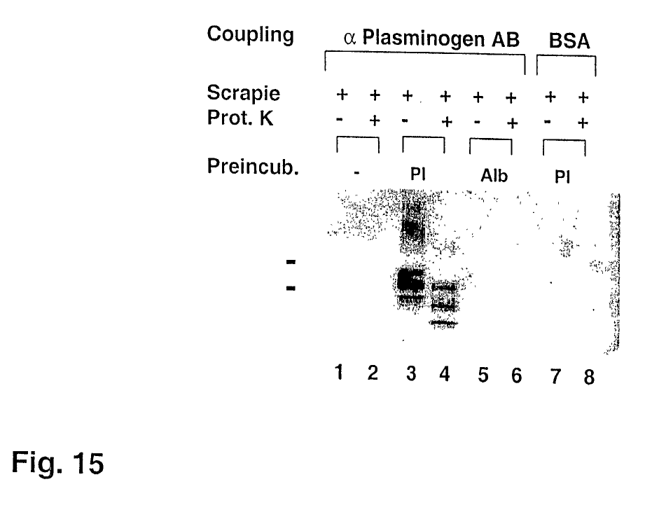

Furthermore it could be shown that PrP^{B} activity of plasminogen is not dependent on the covalent crosslink to the beads by using magnetic beads coated with antibodies directed against plasminogen and preincubated with plasminogen (see FIG. 15, lanes 3-4). There are two negative controls: 1. If beads coated with antibodies against plasminogen are not at all preincubated (see FIG. 15, lanes 1-2) or preicubated with albumin (see FIG. 15, lanes 5-6), the pathogenic isoform of PrP is not bound. 2. If beads coated with albumin are preincubated with plasminogen there is also no binding to the pathogenic isoform of PrP (see FIG. 15, lanes 7-8).

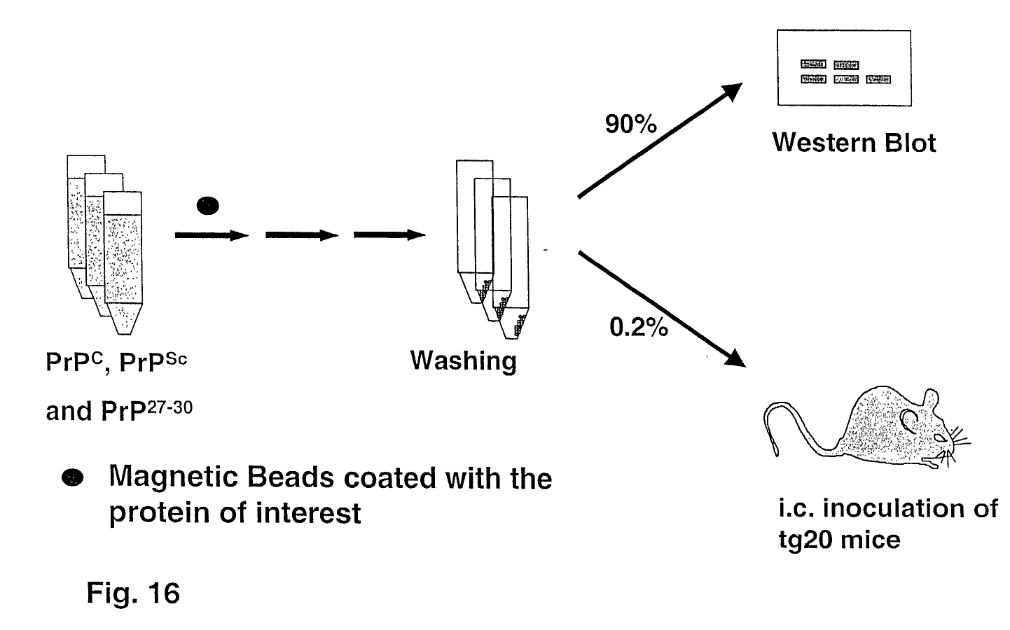

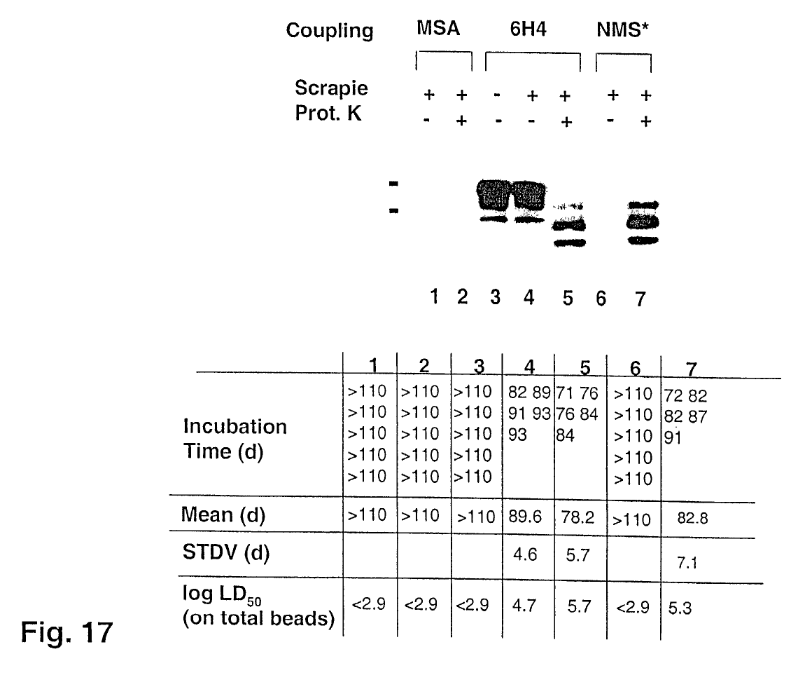

Furthermore it could be shown that at least spPrP^{B} does not only bind the pathogenic PrP but also infectivity. For this purpose we inoculated indicator tga20 mice i.c. with 0.2% of the paramagnetic beads before eluting the other 99% of the beads and performing a western blot (see FIG. 16). The animals that were inoculated with beads that bind the pathogenic PrP did all develop the disease (see FIG. 17, lanes 4, 5 and 7).

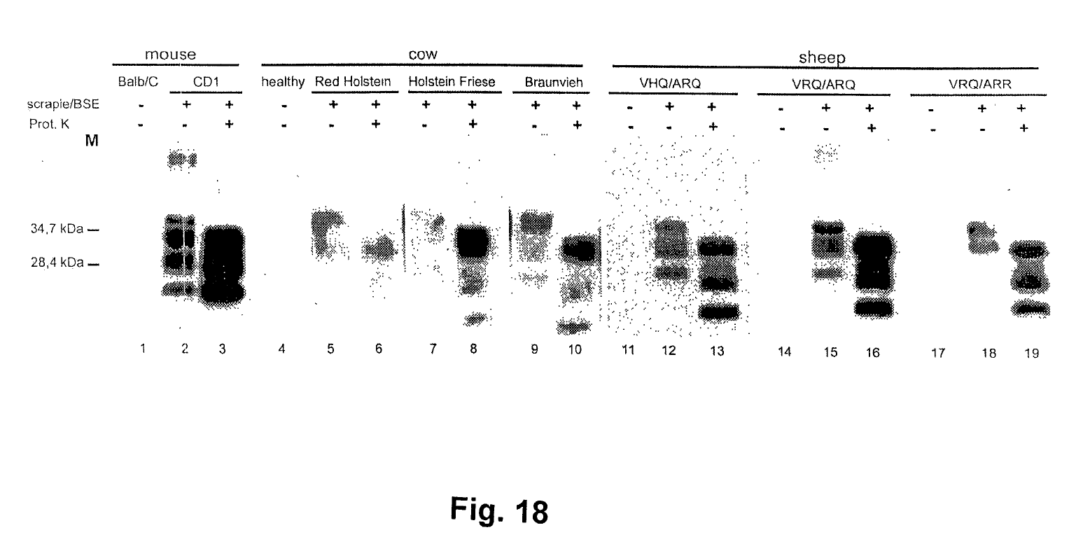

It was also determined whether the interaction between plasminogen and disease-associated prion protein represents a universal feature of spongiform encephalopathies. Human plasminogen (100 μg) was linked to tosyl-activated paramagnetic Dynabeads M-280 (Dynal, Oslo, 1 ml). Brain tissues from a healthy mouse (FIG. 18, lane 1), a scrapie-affected mouse (lanes 2-3), pooled brains of Swiss non-affected cows (lane 4) and brains of BSE-affected cows of various breeds (lanes 5-10) were homogenized as described and tested for the presence of PrP^{Sc}. For this, 50 μg (mouse) or 1 mg (cow) homogenate were incubated with paramagnetic beads coupled to anti-PrP monoclonal antibody 6H4 (data not shown), BSA (negative control; data not shown), or plasminogen. Bead eluates (24 μl) were run on SDS-PAGE (5% stacking -12% resolving) and blotted on nitrocellulose membranes (Schleicher & Schuell, Dassel). For detection of disease-associated PrP, membranes were incubated with 6H4 (Prionics, Zurich)as primary antibody and rabbit-α-mouse IgG₁-HRP (Zymed, San Francisco) as secondary antibody. Membranes were then developed using ECL detection reagents. Signals were recorded on film and/or quantified using a Kodak Image Station. In all cases, plasminogen immobilized to magnetic beads captured PrP^{Sc} from each species when subjected to the precipitation assay. It has been reported that various breeds of sheep are variably susceptible to scrapie. Susceptibility was mapped to polymorphisms at codons 136, 154, 171 within the sheep Prnp gene. Because these polymorphisms occur at the carboxy terminus of the protein and affect basic amino acids, and indirect evidence implies that the carboxy terminus of PrP^{Sc} may participate to the binding to plasminogen, we have investigated whether genetic susceptibility to scrapie in sheep might correlate with the ability of PrP^{Sc} to bind plasminogen. Brain tissue from non-affected and scrapie-affected sheep with the Prnp genotypes at codons 136, 154 and 171 of VHQ/ARQ (FIG. 18, lanes 11-13), VRQ/ARQ (lanes 14-16), and VRQ/ARR (lane 17-19) were homogenized and subjected to the prion affinity assay. Plasminogen precipitated PrP^{Sc} from all sheep genotypes investigated. FIG. 18 eluates from plasminogen beads incubated with brain homogenates were subjected to Western blot analysis. Species and breeds are indicated over the respective lanes. Infection with scrapie or with BSE, and digestion of samples with proteinase K, are marked with “+” and “−” signs. Numbers listed underneath each lane indicate individual cows and sheep of various breeds and Prnp genotypes. Plasminogen beads immobilized PrP^{Sc} in all samples tested.

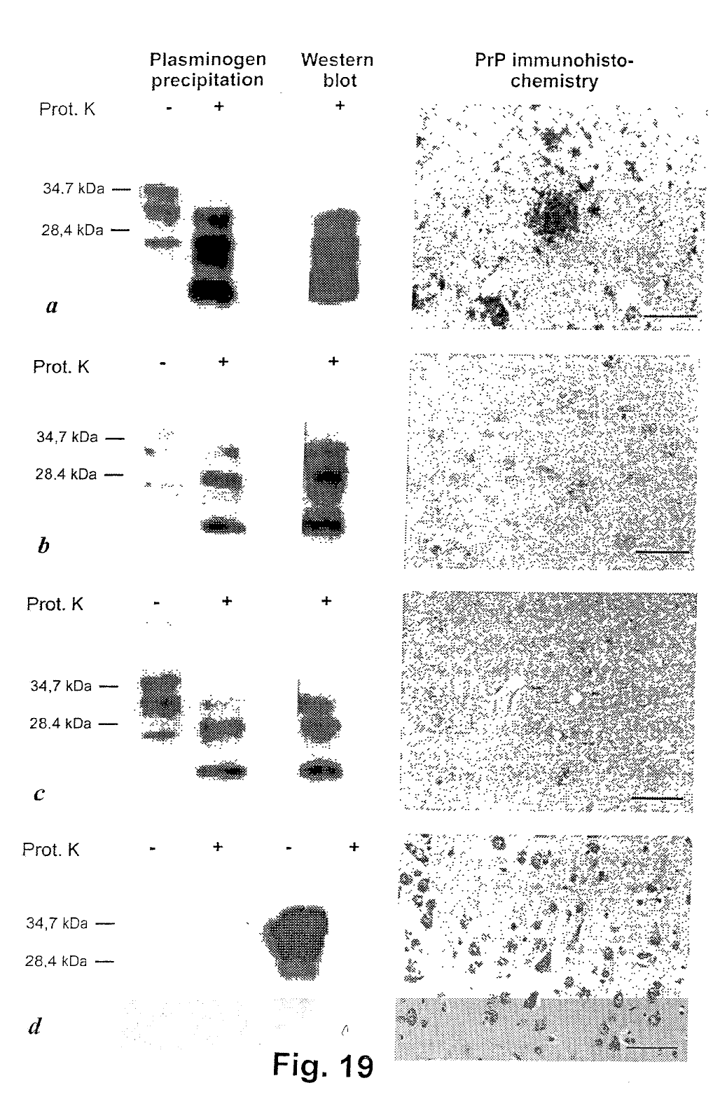

In addition, we tested brain tissues (500 μg) from several patients who died of sporadic Creutzfeldt-Jakob disease, Alzheimer's disease (FIG. 19) and Binswanger's disease (data not shown) with the prion affinity assay. In all assays performed with homogenates of CJD patients, plasminogen was able to precipitate PrP^{Sc}, while no signal was detectable with homogenates of non-CJD patients. Unambiguous positive signals were obtained from cases with plaque-like, patchy-perivacuolar and synaptic pattern of PrP depositions. The intensity of the Prp^{CJD} signals in the precipitation assays correlated closely with histopathological findings (FIG. 19). In FIG. 19 plasminogen precipitated PrP^{CJD} from brain homogenate of three Swiss sCJD patients (a, b, c) exhibiting extensive plaque-like (a) or scant synaptic accumulation (b, c) of PrP^{CJD}. For control we used brain homogenate from a patient suffering from Alzheimer's disease (d). Proteinase K digestion was carried out as indicated with “+” signs over the corresponding lanes. Corresponding brain sections immunostained with antibody 3F4 (available from Dr. Richard Kascksak, Albert Einstein College, The Bronx, N.Y., USA or Draco, Denmark, Botrup) to PrP are displaced on the right side. In each case, the plasminogen-based assay and the Western blot show congruent results. In Alzheimer's disease, PrP^{C} was detectable (−), but not PrP^{Sc} (+). Scale bars are 50 μm.

### EXAMPLES

### Example 1: IAP method

The IAP protocol is the following: Bring the brain tissue in a 15 ml FALCON tube, put it on ice and leave it there for all steps. Add Homogenate Buffer (0.5% DOC/0.5% NP-40 in PBS) to get 10% (w/v) homogenate. Pass the tissue through a 18 gauge needle and a 22 gauge needle by sucking up and down for 15 times each. Centrifuge the homogenate for 30 minutes at 500 g and 4° C. Keep the supernatant. Determine the protein concentration. Centrifuge the homogenate for 30 minutes at 500 g and 4° C. Keep the supernatant. If the protein concentration is higher than 10 mg/ml then bring the homogenate to a protein concentration of 10 mg/ml using the homogenate buffer. Bring the homogenate to a protein concentration of 5 mg/ml and 3% Tween 20/3% NP-40 all in PBS. Add to the tissue homogenate Proteinase K to get a final concentration of 50 μg/ml. Incubate for 60 minutes at 37° C. Add PMSF to get a final concentration of 5 mM. Add 0.25 volumes of IAP buffer (3% Tween 20/3% NP-40 in PBS). Resuspend the magnetic beads (covered with 6H4) according to the protocol described below) thoroughly. Pipette out 100 μl. Remove buffer. Add the homogenate to the beads and incubate the bead-sample mixture with continous mixing for 1.5 hours at room temperature. Collect the beads using the MPC (strong magnet). Wash three times with 1 ml Washing Buffer (2% Tween 20/2% NP-40 in PBS) and once with 1 ml PBS by vortexing for 15 seconds at room temperature and by using the MPC. Spin down the beads, discard the remaining supernatant using again the MPC. Add 24 μl ×Loading Buffer (50 mM Tris pH 6,8; 2% SDS; 0.01% bromphenol blue; 10% glycerol) . Heat to 95° C. for 5 minutes. If the samples are stored at −20° C. then heat them again for 30 seconds at 95° C. before performing SDS-PAGE followed by western Blot: Assemble the glass plates according to the manufacturer's instructions. Prepare in a Falcon tube the appropriate volume of the Resolving Gel (2.1 ml H₂O, 1.5 ml 40% Acrylamid, 1.3 ml 1.5 M Tris pH 8.8, 50 μl 10% SDS, 50 μl 10% Ammoniumpersulfat, 2 μl TEMED). Mix the components in the order shown. Polymerization will begin as soon as the TEMED has been added. Pour the acrylamide solution into the gap between the glass plates. Leave sufficient space for the stacking gel (the length of the comb plus 1 cm). Using a pasteur pipette carefully overlay the acrylamide with water. Place the gel in a vertical position at room temperature. After polymerization is complete (30 minutes), pour off the overlay and wash the top of the gel several times with deionized water to remove any unpolymerized acrylamide. Prepare in a Falcon tube the appropriate volume of the Stacking Gel (1.48 ml H20, 0.25 ml 40% Acrylamid, 0.25 ml 1.0 M Tris pH 6.8, 20 μl 10% SDS, 20 μl 10% Ammoniumpersulfat, 2 μl TEMED). Mix the components in the order shown. Polymerization will begin as soon as the TEMED has been added. Pour the stacking gel solution directly onto the surface of the polymerized resolving gel. Immediately insert a clean Teflon comb into the stacking gel solution, being careful to avoid trapping air bubbles. Place the gel in a vertical position at room temperature. After polymerization is complete (30 minutes), remove the Teflon comb carefully. Mount the gel in the electrophoresis apparatus. Add Running buffer to the top and bottom reservoirs. Remove any (25 mM Tris, 250 mM glycine, 0.1% SDS) bubbles that become trapped at the bottom of the gel between the glass plates. Load 24 μl of each of the samples in a predetermined order into the bottom of the wells (1. well: Low -range marker). Load an equal volume of 1×Gel-loading Buffer into any wells that are unused. Attach the electrophoresis apparatus to an electric power supply (the positive electrode should be connected to the bottom reservoir). Apply 10 V/cm to the gel. After the dye front has moved into the resolving gel (30 minutes), increase the voltage to 14 V (cm and run the gel until the bromophenol blue reaches the bottom of the resolving gel (1 hour). Then turn off the power supply. Cut six sheets of absorbent paper (Whatman 3 MM or equivalent) and one sheet of nitrocellulose to the size of the gel (6 cm×8 cm). If the paper overlaps the edge of the gel, the current will short-circuit the transfer and bypass the gel, preventing efficient transfer. Wet the absorbent paper, the nitrocellulose and the gel by soaking in Transfer (39 mM glycine, 48 mM Tris, 0.037% SDS, 20% methanol) Buffer. On the bottom plate of the apparatus (the anode), assemble the gel, nitrocellulose, and paper in this order:

bottom electrode,

three layers absorbent paper soaked in transfer buffer,

one nitrocellulose membrane soaked in transfer buffer,

polyacrylamide gel slightly wetted with transfer buffer,

three layers absorbent paper soaked in transfer buffer.

Check carefully for air bubbles and gently remove them either by using a gloved hand or by rolling a pipet over the sandwich. Dry any buffer that may surround the gel-paper sandwich. Carefully place the upper electrode (the cathode) on top of the stack. Put a weight on it. Connect the electrodes and commence transfer. Running time is 1 hour with a current of 1 mA/cm². After transfer, disconnect the power source. Carefully disassemble the apparatus. Mark membrane to follow orientation (usually by snipping off lower left-hand corner, the number one lane). Rinse the membrane three times with TBS-T. Add Blocking Buffer (5% (w/v) nonfat dry milk in TBS-T). Incubate at room temperature with agitation for 30 minutes. Rinse the membrane three times with TBS-T. Add to 2.5 μl of mAB 6H4 (2 mg/ml) 12.5 ml of 1% (w/v) nonfat dry milk in TBS-T. Incubate at room temperature with agitation for 1 hour or overnight at 4° C. Remove the membrane from the antibody solution and wash three times for 10 minutes each in TBS-T. Add to 1.25 μl of relativ anti mouce IgG1-HRP 12.5 ml of 1% (w/v) nonfat dry milk in TBS-T. Incubate at room temperature with agitation for 1 hour. Remove the membrane from the antibody solution and wash three times for 15 minutes each in TBS-T. Mix 1 ml of detection solution 1 with 1 ml of detection solution 2 from the ECL Western blotting detection reagents (Amersham Pharmacia Biotech). Incubate for precisely 1 minute at room temperature without agitation. Drain off excess detection reagent by putting the membrane on a absorbent paper. Gently place the membrane, protein side down, on a SaranWrap. Close SaranWrap to form a envelope avoiding pressure on the membrane. Place the membrane, protein side up, in the film cassette. Work as quickly as possible. Switch off the lights and carefully place a sheet of autoradiography film such as (Hyperfilm ECL) on top of the membrane, close the cassette and expose for some seconds (15″, 30″).

### Example 2: PAA method

Couple the protein of interest to magnetic beads: Bring 100 μg of protein into approx. 1 ml of Coupling Buffer (0.1 M borate buffer pH 9.5: dissolve 6.183 g H3BO3 in 800 ml distilled water, Adjust pH to 9.5 using 5 M NaOH and adjust volume to 1000 ml with distilled water; if necessary, change buffer by dialysis). (If coupling was performed in the presence of an excess, 1 mg was used for 1 ml of coupling buffer.) Make a homogeneous suspension of the Dynabeads M-280 Tosylactivated by Dynal using a pipette and by vortexing for approximately 1 min. Pipette out 1 ml of Dynabeads and wash as follows: Place the tube in the DYNAL MPC. Leave to separate for 2 minutes. Remove the supernatant taking care not to disturb the Dynabeads. Remove the tube from the Dynal MPC and resuspend the Dynabeads in PBS. Repeat these steps and resuspend the Dynabeads in the coupling buffer containing the antibodies. Incubate for 24 h at 37° C. with tilt rotation. Place the tube in the magnet for 3 minutes and remove the supernatant. Wash the coated Dynabeads six times: 2 × in PBS/BSA (add 0.1% (w/v) bovine serum albumin (final concentration) to PBS), pH 7.4 for 5 minutes at room temperature; 1 × in Blocking Buffer (0.2 M Tris pH 8.5 with 0.1% (w/v) BSA: dissolve 2.42 g Tris in 80 ml distilled water. Adjust pH to 8.5 using 1 M HCl, add 0.1% BSA and adjust volume to 100 ml with distilled water) for 4 h at 37° C.; 1 × in PBS/BSA, pH 7.4 for 5 minutes at room temperature; 1 × in 1% Tween 20 for 10 minutes; 1 × in PBS/BSA, pH 7.4 for 5 minutes at room temperature. Store the coated Dynabeads in PBS/BSA pH 7.4, 0.02% sodium azide. Then prepare Sample I: Add 1 ml of PAA Buffer (3% NP-40/3% Tween 20 in PBS) to 10 μl of not infected brain homogenate (Protein concentration 5 mg/ml; 0.5% DOC/0.5 NP-40). Then prepare Sample II and III: Add 1 ml of PAA Buffer (3% NP-40/3% Tween 20 in PBS) to 10 μl of infected brain homogenate (Protein concentration 5 mg/ml; 0.5% DOC/0.5 NP-40). Incubate Sample I and Sample II for 30 minutes at 37° C. without PK. Incubate Sample III for 30 minutes at 37° C. with PK at final concentration of 50 μg/ml (add 50 μl of PK 1 mg/ml). Add PMSF to all samples to get a final concentration of 5 mM (add 50 μl of 100 mM PMSF). Resuspend the Magnetic Beads thoroughly. Pipette out 100 μl. Add the beads to the Samples and incubate the bead-sample mixture with continuous mixing for 1.5 hours at room temperature. Collect the beads using the MPC. Wash three times with 1 ml Washing Buffer and once with 1 ml PBS by vortexing for 15 seconds at room temperature and by using the MPC. Spin down the beads, discard the remaining supernatant using again the MPC. Add 24 μl 1 × Loading Buffer. Heat to 95° C. for 5 minutes. If the samples are stored at −20° C. then heat them again for 30 seconds at 95° C. before loading on the gel.

As a positive control of this assay 6H4 is used and as a negative control mouse IgG or mouse albumin (see FIG. 4).

### Example 3

In order to investigate whether a given mouse serum containes IgG that specifically recognize PrP^{Sc} magnetic beads that are already coated by the company DYNAL with sheep antibodies directed against mouse IgGs were used after preincubation with mouse serum. These beads were the first negative control. As a second negative control these beads preincubated with normal mouse serum were used in order to show that IgGs from normal mouse serum do not bind to any form of PrP. Surprisingly the beads alone showed an affinity to PrP^{Sc} but not to PrP²⁷⁻³⁰. Upon preincubation with normal mouse serum also PrP²⁷⁻³⁰ is bound (see FIG. 5). Therefore it was hypothised that the sheep antibodies from DYNAL recognize a molecule that is associated with PrP^{Sc} but digested away after PK-treatment. As PrP²⁷⁻³⁰ is bound upon preincubation with normal mouse serum, this serum might contain the molecule with affinity to PrP^{Sc}.

### Example 4

The beads coupled to total mouse serum proteins did not show any affinity to any form of PrP. However, if the coupling of the total serum was performed in the presence of an excess of protein the beads showed the same binding to PrP²⁷⁻³⁰ as the monoclonal antibody 6H4 whereas the beads that were coupled in the presence of an excess of albumine still did not show any affinity to any form of PrP (see FIG. 6). Though it was not possible to measure any difference of the coupling efficency of the two conditions it might be that offering an excess of proteins causes a sponge on the surface of the beads that binds PrP²⁷⁻³⁰.

### Example 5

We also checkled whether PK-treated brain homogenate might enhance the binding as in the case of bound PrP²⁷⁻³⁰ total PK-digested brain homogenate is present: the addition of PK-digested brain homogenate from wild-type C57BL/6 mice or Prnp % mice allowed to bind PrP^{Sc} in addition to PrP²⁷⁻³⁰; the addition of inactive PK had no influence on the binding activity (see FIG. 7).

### Example 6

If coupled in the presence of an excess the activity of binding PrP²⁷⁻³⁰ was also found in the serum of man, sheep, cow and in the serum of terminally scrapie-sick C57BL/6 mice (data not shown).

### Example 7

The template-directed refolding hypothesis predicts that PrP^{C} and PrP^{Sc} form heterodimers during the conversion process. Therefore we investigated whether PrP^{B} is identical with PrP^{C}. However, when coupling in excess PrP^{B} activity was present in the serum of Prnp % mice at levels similar to those of wild-type mice, implying that PrP^{C} does not contribute to the binding activity (se FIG. 8).

### Example 8

If PrP^{B} activity is not only caused by the special couspling conditions, it should be possible to “purify” it by fractionating mouse serum by differential ammonium sulfate precipitation. Indeed, it was possible to precipitate PrP^{B} at an ammonium sulfate saturation below 50% whereby coupling of each fraction was performed in the presence of an excess of protein (see FIG. 9). While purified rabbit immunoglobulins against total mouse serum did not contain Prp^{B} (data not shown), they efficiently bound PrP²⁷⁻³⁰ upon preincubation with full mouse serum or with proteins precipitating between 25% and 50% ammonium sulfate saturation. Preincubation with proteins precipitating between 75% and 100% ammonium sulfate saturation did not lead to Prp^{B} activity (see FIG. 10). This finding is important as it shows that the PrP^{B} activity is a property of one or more serum proteins independent of the covalent crosslink to the surface of the beads.

### Example 9

As the ammonium sulfate fractionation worked with human serum as well (data not shown), 58 fractions of human plasma were obtained by chromatography and differential precipitation and tested for binding activity to form an idea of the identity of PrP^{B}. All fractions were not coupled in the presence of an excess of proteins. Therefore the results can directly be compared with 6H4 or mouse IgG. 20 fractions tested positive: Plasminogen, fibrinogen, antithrombin III, antithrombin III heparin complex, C1 esterase inhibitor, factor IX and several fractions containing protein mixtures (see FIG. 11). Purified plasminogen and also purified fibrinogen bound PrP^{Sc} in addition to PrP²⁷⁻³⁰ (see FIG. 12). Out of the 38 fractions that tested negative, 6 contained purified proteins: Prothrombin complex concentrate, albumin, activated prothrombin complex concentrate, factor XIII and thrombin.

### Example 10

As calcium is an important cofactor in the coagulation cascade it was investigated whether PrP^{B} activity is still intact if coagulation is inhibited by complexing calcium. In the presence of 10 mM EDTA the pathogenic PrP^{Sc} and PrP²⁷⁻³⁰ were still bound by plasminogen but only PrP²⁷⁻³⁰ by fibrinogen (FIG. 13). At least in the case of plasminogen this finding speaks against the possibility that the PrP^{B} activity is due to unspecific coagulation.

### Example 11

Because Prp^{B} selectively interacts with the pathogenic PrP but not with PrP^{C}, interaction may be conformation-specific. When the assay was carried out in the presence of 6 M urea the fraction containing purified plasminogen didn't bind PrP^{Sc} nor PrP²⁷⁻³⁰; under these conditions PrP^{Sc} becomes protease-sensitive (FIG. 14). As the conformation of PrP^{Sc} is thought to be responsible for the PK resistancy we conclude from this experiment that the interaction of plasminogen and PrP^{Sc} is conformation-dependant.

### Example 12

Furthermore it could be shown that PrP^{B} activity of plasminogen is not dependent on the covalent crosslink to the beads by using magnetic beads coated with antibodies directed against plasminogen and preincubated with plasminogen (FIG. 15).

### Example 13

Furthermore it could be shown that at least spPrP^{B} does not only bind the pathogenic PrP but also infectivity. For this purpose we inoculated indicator tga20 mice i.c. with 0.2% of the paramagnetic beads before eluting and performing a western blot. The animals that were inoculated with beads that bind the pathogenic PrP did all develop the disease (FIG. 16, FIG. 17).

### Example 14

The prior art offers a large number of options for determining and characterizing the binding characteristics of a given peptide or protein to a certain target. Binding assay for determining the selectivity of a PrPSc specific binding partner using solid state-bound technologies includes e.g. microtiter plate formats, paramagnetic beads, non-magnetic beads, plasmon surface resonance, interferometry, coincidence detection, mass spectrometry/mass spectroscopy, electrospray analysis, and combinations thereof. For use in the present invention the following two approaches are preferred.

1. The peptides or protein or fragments thereof to be tested are coupled to a solid phase material:

a. Use micro particles such as magnetic beads as solid phase and perform immunopreciptiation.

Couple the peptides to magnetic beads. Incubate the beads with PrPSc, PrP27-30 or PrPC. Detect whether the prion protein has bound to the peptides either by Western blot analysis or by microparticle immunoassay.

b. Use surface of a micro titer plate as solid phase and perform ELISA.

Coat the surface of the wells of a micro titer plate with the peptides. Add PrPSc, PrP27-30 or PrPC to the wells. Detect whether the prion protein has bound to the peptides by ELISA.

2. PrPSc (or PrP27-30) and PrPC are coupled to a solid phase material:

a. Use micro particles such as magnetic beads as solid phase and perform immunopreciptiation.

Couple PrPSc (or PrP27-30) and PrPC, respectively, to magnetic beads. Incubate the beads with the peptides or protein fragments to be tested. Detect whether the peptides have bound to the PrPSc but not to PrPC either by Western blot analysis or by microparticle immunoassay.

b. Use surface of a micro titer plate as solid phase and perform ELISA.

Coat the surface of the wells of a micro titer plate with PrPSc (or PrP27-30) and PrPC, respectively. Add peptides or protein fragments to be tested to the wells. Detect whether the peptides have bound to PrPSc but not to PrPC by ELISA.

### Example 15

The prior art offers many possibilities to determine and detect certain parts of a protein which are involved in specific binding of the protein to a certain target.

Method to identify suitable fragments of plasminogen as PrP^{Sc} specific binding partners includes e.g. forward genetic selection using phage display, ribosomal display, bacterial protein fragment affinity assay, and combinations or derivations thereof. Accordingly, those parts of plasminogen that are involved in the specific binding to PrP^{Sc} can be determined as follows:

1. Produce peptide libraries of plasminogen fragments and/or mutants displayed on phage, expose them to a solid phase coated with the pathological prion protein and select for the clones with the maximal binding affinity to PrPSc but minimal affinity to PrPC.

2. Express fragments and/or mutants of plasminogen in a host cell such as bacteria, yeast, fungi or eukaryotic cells, purify the peptides, label them and test them for binding activity.

3. Express fusion proteins with plasminogen fragments and/or mutants in a host cell and test them for PrPSc affinity in a binding assay.

References

Aguzzi, A. (1997). Neuro-immune connection in spread of prions in the body? The Lancet 349, 742-743.

Aguzzi, A. (1998). Protein conformation dictates prion strain. Nat Med 4, 1125-6.

Aguzzi, A., Blattler, T., Klein, M. A., Raber, A. J., Hegyi, I., Frigg, R., Brandner, S., and Weissmann, C. (1997). Tracking prions: the neurografting approach. Cell Mol Life Sci 53, 485-95.

Aguzzi, A., and Collinge, J. (1997). Post-exposure prophylaxis after accidental prion inoculation. Lancet 350, 1519-20.

Aguzzi, A., and Weissmann, C. (1997). Prion research: the next frontiers. Nature 389, 795-798.

Aguzzi, A., and Weissmann, C. (1998). Spongiform encephalopathies. The prion's perplexing persistence. Nature 392, 763-4.

Aguzzi, A., and Weissmann, C. (1996). Spongiform encephalopathies: a suspicious signature. Nature 383, 666-7.

Bernoulli, C., Siegfried, J., Baumgartner, G., Regli, F., Rabinowicz, T., Gajdusek, D. C., and Gibbs, C. J. (1977). Danger of accidental person-to-person transmission of Creutzfeldt-Jakob disease by surgery [letter]. Lancet 1, 478-479.

Blattler, T., Brandner, S., Raeber, A. J., Klein, M. A., Voigtlander, T., Weissmann, C., and Aguzzi, A. (1997). PrP-expressing tissue required for transfer of scrapie infectivity from spleen to brain. Nature 389, 69-73.

Brandner, S., Isenmann, S., Raeber, A., Fischer, M., Sailer, A., Kobayashi, Y., Marino, S., Weissmann, C., and Aguzzi, A. (1996). Normal host prion protein necessary for scrapie-induced neurotoxicity. Nature 379, 339-43.

Bruce, M. E., Will, R. G., Ironside, J. W., McConnell, I., Drummond, D., Suttie, A., McCardle, L., Chree, A., Hope, J., Birkett, C., Cousens, S., Fraser, H., and Bostock, C. J. (1997). Transmissions to mice indicate that ‘new variant’ CJD is caused by the BSE agent [see comments]. Nature 389, 498-501.

Chazot, G., Broussolle, E., Lapras, C., Blattler, T., Aguzzi, A., and Kopp, N. (1996). New variant of Creutzfeldt-Jakob disease in a 26-year-old French man [letter]. Lancet 347, 1181.

Duffy, P., Wolf, J., Collins, G., DeVoe, A. G., Streeten, B., and Cowen, D. (1974). Possible person-to-person transmission of Creutzfeldt-Jakob disease. N Engl J Med 290, 692-3.

Fraser, H., Brown, K. L., Stewart, K., McConnell, I., McBride, P., and Williams, A. (1996). Replication of Scrapie in Spleens of Scid Mice Follows Reconstitution With Wild-Type Mouse Bone Marrow. Journal of General Virology 77, 1935-1940.

Gibbs, C. J., Jr., Joy, A., Heffner, R., Franko, M., Miyazaki, M., Asher, D. M., Parisi, J. E., Brown, P. W., and Gajdusek, D. C. (1985). Clinical and pathological features and laboratory confirmation of Creutzfeldt-Jakob disease in a recipient of pituitary-derived human growth hormone. N Engl J Med 313, 734-8.

Hill, A. F., Butterworth, R. J., Joiner, S., Jackson, G., Rossor, M. N., Thomas, D. J., Frosh, A., Tolley, N., Bell, J. E., Spencer, M., King, A., Al-Sarraj, S., Ironside, J. W., Lantos, P. L., and Collinge, J. (1999). Investigation of variant Creutzfeldt-Jakob disease and other human prion diseases with tonsil biopsy samples. Lancet 353, 183-9.

Hill, A. F., Desbruslais, M., Joiner, S., Sidle, K. C., Gowland, I., Collinge, J., Doey, L. J., and Lantos, P. (1997). The same prion strain causes vCJD and BSE [letter] [see comments]. Nature 389, 448-50.

Hill, A. F., Zeidler, M., Ironside, J., and Collinge, J. (1997). Diagnosis of new variant Creutzfeldt-Jakob disease by tonsil biopsy. Lancet 349, 99.

Hilton, D. A., Fathers, E., Edwards, P., Ironside, J. W., and Zajicek, J. (1998). Prion immunoreactivity in appendix before clinical onset of variant Creutzfeldt-Jakob disease [letter]. Lancet 352, 703-4.

Kitamoto, T., Muramoto, T., Mohri, S., Doh ura, K., and Tateishi, J. (1991). Abnormal isoform of prion protein accumulates in follicular dendritic cells in mice with Creutzfeldt-Jakob disease. J. Virol. 65, 6292-6295.

Klein, M. A., Frigg, R., Flechsig, E., Raeber, A. J., Kalinke, U., Bluethmann, H., Bootz, F., Suter, M., Zinkernagel, R. M., and Aguzzi, A. (1997). A crucial role for B cells in neuroinvasive scrapie. Nature 390, 687-90.

Klein, M. A., Frigg, R., Raeber, A. J., Flechsig, E., Hegyi, I., Zinkernagel, R. M., Weissmann, C., and Aguzzi, A. (1998). PrP expression in B lymphocytes is not required for prion neuroinvasion. Nat Med 4, 1429-33.

Korth, C., Stierli, B., Streit, P., Moser, M., Schaller, O., Fischer, R., Schulz-Schaeffer, W., Kretzschmar, H., Raeber, A., Braun, U., Ehrensperger, F., Hornemann, S., Glockshuber, R., Riek, R., Billeter, M., Wuthrich, K., and Oesch, B. (1997). Prion (PrP^{Sc})-specific epitope defined by a monoclonal antibody. Nature 390, 74-7.

Lasmezas, C. I., Cesbron, J. Y., Deslys, J. P., Demaimay, R., Adjou, K. T., Rioux, R., Lemaire, C., Locht, C., and Dormont, D. (1996). Immune system-dependent and -independent replication of the scrapie agent. J Virol 70, 1292-5.

O'Rourke, K. I., Huff, T. P., Leathers, C. W., Robinson, M. M., and Gorham, J. R. (1994). SCID mouse spleen does not support scrapie agent replication. J. Gen. Virol. 75, 1511-1514.

Raeber, A. J., Klein, M. A., Frigg, R., Flechsig, E., Aguzzi, A., and Weissmann, C. (1999). PrP-dependent association of prions with splenic but not circulating lymphocytes of scrapie-infected mice. EMBO J 18, 2702-2706.

Schreuder, B. E., van Keulen, L. J., Smits, M. A., Langeveld, J. P., and Stegeman, J. A. (1997). Control of scrapie eventually possible? Vet Q 19, 105-13.

Schreuder, B. E., van Keulen, L. J., Vromans, M. E., Langeveld, J. P., and Smits, M. A. (1998). Tonsillar biopsy and PrPSc detection in the preclinical diagnosis of scrapie. Vet Rec 142, 564-8.

Vankeulen, L. J. M., Schreuder, B. E. C., Meloen, R. H., Mooijharkes, G., Vromans, M. E. W., and Langeveld, J. P. M. (1996). Immunohistochemical Detection of Prion Protein in Lymphoid Tissues of Sheep With Natural Scrapie. Journal of Clinical Microbiology 34, 1228-1231.

Weber, T., and Aguzzi, A. (1997). The spectrum of transmissible spongiform encephalopathies. Intervirology 40, 198-212.

Weissmann, C., and Aguzzi, A. (1997). Bovine spongiform encephalopathy and early onset variant Creutzfeldt-Jakob disease. Curr Opin Neurobiol 7, 695-700.

Wells, G. A., Hawkins, S. A., Green, R. B., Austin, A. R., Dexter, I., Spencer, Y. I., Chaplin, M. J., Stack, M. J., and Dawson, M. (1998). Preliminary observations on the pathogenesis of experimental bovine spongiform encephalopathy (BSE): an update. Vet Rec 142, 103-6.

Will, R., Cousens, S., Farrington, C., Smith, P., Knight, R., and Ironside, J. (1999). Deaths from variant Creutzfeldt-Jakob disease. Lancet 353, 9157-9158.

Will, R., Ironside JW, Zeidler M, Cousens SN, Estibeiro K, Alperovitch A, Poser S, Pocchiari M, Hofman A, and Smith (1996). A new variant of Creutzfeldt-Jakob disease in the UK. Lancet 347, 921-925.

1. Factor which selectively interacts with a PrPSc but not with PrPc.

2. Factor according to claim 1 which is selected from plasminogen, fragments of plasminogen and derivatives thereof.

3. Factor according to any of claims 1 or 2, characterized in that it interacts with the carboxy terminus of PrPSc.

4. Factor according to any of claims 1 to 3, characterized in that it is capable of interacting with PrPSc of different species.

5. Composition comprising a PrPSc and a factor according to any of claims 1 to 4.

6. Composition according to claim 5, wherein PrPSc is bound to the factor.

7. Composition according to claim 6, wherein PrPSc is noncovalently bound to the factor.

8. A carrier comprising a factor according to any of claims 1 to 4 and/or a composition according to any of claims 5 to 7.

9. Carrier according to claim 8 which is selected from magnetic beads, filter stripes, microtiter plates, non-magnetic beads, plasmon surface resonance plates, microarray plates, liquid carriers undergoing phase transition to solid, and combination thereof.

10. Ligand which specifically interacts with a composition according to any of claims 5 to 7.

11. Diagnostic kits containing a factor according to any of claims 1 to 4 and/or a composition according to any of claims 5 to 7 and/or a carrier according to any of claims 8 and 9 and/or a ligand according to claim 10, optionally together with further components such as buffers, reagents for the detection and working instructions.

12. Pharmaceutical composition comprising a factor according to any of claims 1 to 4 and/or a ligand according to claim 10.

13. A process for detecting a PrPSc in a sample, characterized in that the sample is contacted with a factor according to any of claims 1 to 4 and/or a carrier according to claims 8 or 9 and/or a ligand according to claim 10.

14. A process for removing PrPSc from biological material, comprising the step of contacting the material with a factor according to any of claims 1 to 4 and/or a carrier according to any of claims 8 or 9 and/or a ligand according to claim 10.

15. Method for diagnosing human transmissible spongiform encephalopathies and prion encephalopathies of animals, characterized in that the material of the organism to be tested in brought into contact with a factor according to any of claims 1 to 4 and/or a carrier according to any of claims 8 to 9 and/or a ligand according to claim 10.

16. Use of a factor according to any of claims 1 to 4 and/or a composition according to any of claims 5 to 7 and/or a carrier according to any of claims 8 or 9 and/or a ligand according to claim 10 for the diagnosis of human transmissible spongiform encephalopathies or prion encephalopathies of animals.

17. Use of a factor according to any of claims 1 to 4 and/or a composition according to any of claims 5 to 7 and/or a carrier according to any of claims 8 or 9 and/or a ligand according to claim 10 for removing PrPSc from and/or inactivating PrPc in a biological material.
